(19) United States
(12) Patent Application Publication
(10) Pub. No.: US 2011/0287424 A1
(43) Pub. Date: Nov. 24, 2011

(54) METHYLATION-SPECIFIC COMPETITIVE ALLELE-SPECIFIC TAQMAN POLYMERASE CHAIN REACTION (CAST-PCR)

(75) Inventor: Caifu CHEN, Palo Alto, CA (US)

(73) Assignee: LIFE TECHNOLOGIES CORPORATION, Carlsbad, CA (US)

(21) Appl. No.: 13/098,220

(22) Filed: Apr. 29, 2011

Related U.S. Application Data
(63) Continuation-in-part of application No. 12/748,329, filed on Mar. 26, 2010.
Application No. 12/748,329 is a continuation-in-part of application No. 12/641,321, filed on Dec. 17, 2009.
(60) Provisional application No. 61/329,529, filed on Apr. 29, 2010.
(60) Provisional application No. 61/258,582, filed on Nov. 5, 2009.
(60) Provisional application No. 61/253,501, filed on Oct. 20, 2009.
(60) Provisional application No. 61/251,623, filed on Oct. 14, 2009.
(60) Provisional application No. 61/186,775, filed on Jun. 12, 2009.
(60) Provisional application No. 61/164,230, filed on Mar. 27, 2009.

(51) Int. Cl. C12Q 1/68

(52) U.S. Cl. 435/6.11

(57) ABSTRACT

In some embodiments, the present inventions relates generally to compositions, methods and kits for use in discriminating between different methylated and/or unmethylated nucleic acid loci. In certain embodiments, the inventions provides for detecting or quantitating undifferentiated embryonic stem cells in a population of differentiated cells. The invention is also useful for discriminating between fetal versus maternal cells, or healthy versus infected cells, or normal versus cancerous cells, or detecting reduction in viral load or measuring therapeutic efficiency in a patient, and more.

### CROSS REFERENCE TO RELATED APPLICATIONS

This application claims benefit of priority under 35 U.S.C. 119(e) from the U.S. Provisional Application No. 61/329,529 filed 29 Apr. 2010, which is incorporated hereby in its entirety. This application is also a continuation-in-part application of U.S. application Ser. No. 12/748,329, filed 26 Mar. 2010, which in turn is a continuation-in-part application of U.S. application Ser. No. 12/641,321, filed Dec. 17, 2009 and claims the benefit of priority under 35 U.S.C. 119(e) to U.S. Provisional Application Ser. Nos. 61/258,582, filed Nov. 5, 2009; 61/253,501, filed Oct. 20, 2009; 61/251,623, filed Oct. 14, 2009; 61/186,775, filed Jun. 12, 2009; and 61/164,230, filed Mar. 27, 2009, all of which are incorporated herein by reference in their entireties.

### BACKGROUND

Single nucleotide polymorphisms (SNPs) are the most common type of genetic diversity in the human genome, occurring at a frequency of about one SNP in 1,000 nucleotides or less in human genomic DNA (Kwok, P-Y, Ann Rev Genome Hum Genet. 2001, 2: 235-258). SNPs have been implicated in genetic disorders, susceptibility to different diseases, predisposition to adverse reactions to drugs, and for use in forensic investigations. Thus, SNP (or rare mutation) detection provides great potentials in diagnosing early phase diseases, such as detecting circulating tumor cells in blood, for prenatal diagnostics, as well as for detection of disease-associated mutations in a mixed cell population.

Numerous approaches for SNP genotyping have been developed based on methods involving hybridization, ligation, or DNA polymerases (Chen, X., and Sullivan, P F, The Pharmacogeonomics Journal 2003, 3, 77-96). For example, allele-specific polymerase chain reaction (AS-PCR) is a widely used strategy for detecting DNA sequence variation (Wu D Y, Ugozzoli L, Pal B K, Wallace R B., Proc Natl Acad Sci USA 1989; 86:2757-2760). AS-PCR, as its name implies, is a PCR-based method whereby one or both primers are designed to anneal at sites of sequence variations which allows for the ability to differentiate among different alleles of the same gene. AS-PCR exploits the fidelity of DNA polymerases, which extend primers with a mismatched 3′ base at much lower efficiency, from 100 to 100,000 fold less efficient, than that with a matched 3′ base (Chen, X., and Sullivan, P F, The Pharmacogeonomics Journal 2003; 3:77-96). The difficulty in extending mismatched primers results in diminished PCR amplification that can be readily detected.

The specificity and selectivity of AS-PCR, however, is largely dependent on the nature of exponential amplification of PCR which makes the decay of allele discriminating power rapid. Even though primers are designed to match a specific variant to selectively amplify only that variant, in actuality significant mismatched amplification often occurs. Moreover, the ability of AS-PCR to differentiate between allelic variants can be influenced by the type of mutation or the sequence surrounding the mutation or SNP (Ayyadevara S, Thaden J J, Shmookler Reis R J., Anal Biochem 2000; 284:11-18), the amount of allelic variants present in the sample, as well as the ratio between alternative alleles. Collectively, these factors are often responsible for the frequent appearance of false-positive results, leading many researchers to attempt to increase the reliability of AS-PCR (Orou A, Fechner B, Utermann G, Menzel H J., Hum Mutat 1995; 6:163-169)(Imyanitov E N, Busby K G, Suspitsin E N, Kuligina E S, Belogubova E V, Grigoriev M Y, et al., Biotechniques 2002; 33:484-490)(McKinzie P B, Parsons B L. Detection of rare K-ras codon 12 mutations using allele-specific competitive blocker PCR. Mutat Res 2002; 517:209-220)(Latorra D, Campbell K, Wolter A, Hurley J M., Hum Mutat 2003; 22:79-85).

In some cases, the selectivity of AS-PCR has been increased anywhere from detection of 1 in 10 alleles to 1 in 100,000 alleles by using SNP-based PCR primers containing locked nucleic acids (LNAs) (Latorra, D., et al., Hum Mut 2003, 2:79-85; Nakiandwe, J. et al., Plant Method 2007, 3:2) or modified bases (Koizumi, M. et al. Anal Biochem. 2005, 340:287-294). However, these base “mimics” or modifications increase the overall cost of analysis and often require extensive optimization.

Another technology involving probe hybridization methods used for discriminating allelic variations is TaqMan® genotyping. However, like AS-PCR, selectivity using this method is limited and not suitable for detecting rare (1 in ≧1,000) alleles or mutations in a mixed sample.

Human and/or other embryonic stem cells (hESCs or ESCs) are derived from pre-implantation blastocysts and may retain the remarkable ability to differentiate into multiple different tissues and to self-renew this property in vitro. These abilities make ESCs very interesting to the scientific and clinical communities because they may be a renewable source of a wide variety of tissues which can be used for regenerative medicine, in drug discovery and toxicity testing.

While significant progress has been made to differentiate ESCs into various cell types, there are several major obstacles that can hinder clinic trials in human and other patients. For example, during the cell type-specific differentiation of hESCs, a small fraction of undifferentiated hESCs frequently remain mixed with the differentiated cells. These undifferentiated hESCs pose serious cancer risk by forming tumors after being transplanted into patients. Also, regulatory agencies require that level of contaminating material remains below a predetermined threshold. Therefore, it is important to be able to detect and quantify undifferentiated hESCs in samples prior to use.

Although not formally recognized as a single nucleotide polymorphism, methylation of specific nucleotides (for example, methylcytosine or ^{m}C) does impart different characteristics compared to their un-methylated state and may be considered a special kind of single nucleotide polymorphism. DNA methylation is an important epigenetic modification that plays critical roles in cellular differentiation, development, and disease. Hypermethylation is strongly associated with heterochromatin and transcriptional silencing. In particular, correct DNA methylation is critical for X-inactivation, imprinting, and silencing of specific genomic elements, such as transposons. Derangements in DNA methylation patterns have been associated with dysregulation of gene expression in cancer cells, particularly down-regulation of genes with tumor suppressor functions by hypermethylation of their promoter regions.

In mammals, the predominant form of DNA methylation occurs symmetrically on the cytosine residues on both strands of CpG dinucleotides, although there is evidence that cytosine methylation is not limited to those in CpG sequences. About 70%-80% of CpG sites in mammalian cells are methylated, but both the CpG sites and their degrees of methylation are unevenly distributed in the genome. CpG dinucleotides are largely concentrated in small regions termed “CpG islands”, which are found within the promoters of ˜70% of human genes. CpGs located in promoter-associated CpG islands tend to be unmethylated, but a subset are differentially methylated in specific tissues or during the course of development, and result in transcriptional repression of the adjacent genes (from Laurent, L., et al., Genome Res. 20: 320-331 (2010)).

Recently, Laurent, et al. (Laurent, L., et al., Genome Res. 20: 320-331 (2010)) and Lister, et al. (Lister, R., et al, Nature (2009), vol. 462: pp. 315-322), the disclosures of which are hereby incorporated by reference in their entireties, presented a whole-genome comparative view of DNA methylation using bisulfite sequencing of three cultured cell types representing progressive stages of differentiation: human embryonic stem cells (hESCs), a fibroblastic differentiated derivative of the hESCs (E-fibroblasts), and neonatal fibroblasts. They compared their methylation “maps” with a methylome map of a fully differentiated adult cell type, namely mature peripheral blood mononuclear cells (monocytes). As a consequence, they observed many notable common and cell-type-specific features among all cell types. They report, “Promoter hypomethylation (both CG and CA) and higher levels of gene body methylation were positively correlated with transcription in all cell types. Exons were more highly methylated than introns, and sharp transitions of methylation occurred at exon-intron boundaries, suggesting a role for differential methylation in transcript splicing. Developmental stage was reflected in both the level of global methylation and extent of non-CpG methylation, with hESC highest, fibroblasts intermediate, and monocytes lowest. Differentiation-associated differential methylation profiles were observed for developmentally regulated genes, including the HOX clusters, other homeobox transcription factors, and pluripotence-associated genes such as POU5F1, TCF3, and KLF4.”

Based on these findings, many genes and genomic DNA regions have been identified between hESCs and differentiated cells which have distinct DNA methylation patterns. For example, Laurent, et al found that the degree of global DNA methylation was inversely correlated with differentiation status. The highest level of methylation was seen in the undifferentiated hESCs, suggesting that a global reduction in DNA methylation occurs during differentiation. On the other hand, their data also identified regions of ESC genome that were lacking in methylation while the same regions were methylated in the differentiated cells. Similarly, these distinct methylation patterns can be used to distinguish between any different source of cells, i.e., cancerous v. normal cells; fetal v. maternal cells; healthy v. infected cells and more. Therefore, these identified regions can be used as methylation markers to distinguish between undifferentiated hESCs and their differentiated (somatic) cell counterparts.

Recently, Pollex et al. have reported a method for detecting methylcytosine in various RNAs (Pollex et al., Cold Spring Harb. Protoc. 2010; doi:10.1101/pdb.prot5505 (available at: http://cshprotocols.cshlp.org/cgi/content/full/2010/10/pdb.prot5505 (accessed on 26 Apr. 2011): Post-transcriptional RNA modifications are a characteristic feature of noncoding RNAs and have been described for ribosomal RNAs (rRNAs), transfer RNAs (tRNAs), and various other small RNAs. However, the biological function of most of these modifications remains uncharacterized. Cytosine-5 methylation (5mC) has been detected in abundant and long-lived RNA molecules such as rRNAs and tRNAs, but, because of technical limitations, the occurrence of base-methylated cytosines in other RNAs is not known. To facilitate the detection of RNA methylation, Pollex et al. have established a method for analyzing base-methylated cytosines in RNA using bisulfite sequencing. Treatment of RNA with bisulfite causes the chemical deamination of nonmethylated cytosines to uracil, while methylated cytosines remain unaffected. cDNA synthesis followed by PCR amplification and detection allows investigators to reproducibly and quantitatively distinguish unmethylated cytosines (as thymines) from methylated cytosines in RNAs. Thus, there is need to improve upon methylation detection techniques for various purposes.

### SUMMARY

Competitive allele-specific TaqMan PCR (castPCR) is capable of detecting rare mutations in 1 out of 10,000,000 copies. See for example, U.S. application Ser. Nos. 12/641,321, filed Dec. 17, 2009; and 12/748,329, filed Mar. 26, 2010; and U.S. Provisional Application Nos. 61/258,582, filed Nov. 5, 2009; 61/253,501, filed Oct. 20, 2009; 61/251,623, filed Oct. 14, 2009; 61/186,775, filed Jun. 12, 2009; 61/164,230, filed Mar. 27, 2009; and 61/138,52, filed Dec. 17, 2008, all of which are incorporated herein by reference in their entireties.

Here we describe methods of methylation-specific castPCR which can be used to detect rare undifferentiated hESCs based on their distinct DNA methylation patterns.

Methylation-specific cast PCR is a novel tool for stem cell therapy products. The use of methylation-specific castPCR for the detection of rare undifferentiated hESCs provides several advantages over conventional methods. Some of the advantages for using methylation-specific castPCR methods to detect rare undifferentiated hESCs in stem cell therapy products include, for example, (a) high specificity; (b) high sensitivity; and (c) the speed at which the assays can be performed.

The methods of the invention are also useful for detecting or discriminating between fetal versus maternal cells, or healthy versus infected cells, or normal versus cancerous cells, or detecting reduction in viral load or measuring therapeutic efficiency in a patient, and more.

In one aspect, the invention discloses a method for detecting at least one first unmethylated cytosine allelic variant of a target sequence in a nucleic acid sample suspected of also comprising at least one methylated cytosine allelic variant of the target sequence, comprising: bisulfite converting an aliquot of a nucleic acid sample; forming a reaction mixture by combining: the bisulfite converted nucleic acid sample aliquot; a first allele-specific primer, wherein an unmethylated cytosine allele-specific nucleotide portion of the first allele-specific primer is complementary to the first uracil-containing allelic variant of the target sequence; a first allele-specific blocker probe, wherein a cytosine allele-specific nucleotide portion of the first allele-specific primer is complementary to the first methylcytosine-containing allelic variant of the target sequence, and wherein the first allele-specific blocker probe comprises a minor groove binder; a first locus-specific primer that is complementary to a region of the target sequence that is 3′ from the first allelic variant and on the opposite strand; and a first detector probe; carrying out an amplification reaction; and detecting the first amplicon by detecting a change in a detectable property of the first detector probe, thereby detecting the first unmethylated cytosine allelic variant of the target gene in the nucleic acid sample.

It is to be understood that both the foregoing general description and the following detailed description are exemplary and explanatory only and are not restrictive of the invention, as claimed.

The accompanying drawings, which are incorporated in and constitute a part of this specification, illustrate several exemplary embodiments of the disclosure and together with the description, serve to explain certain teachings.

### BRIEF DESCRIPTION OF THE DRAWINGS

FIG. 1 is a schematic describing CastPCR.

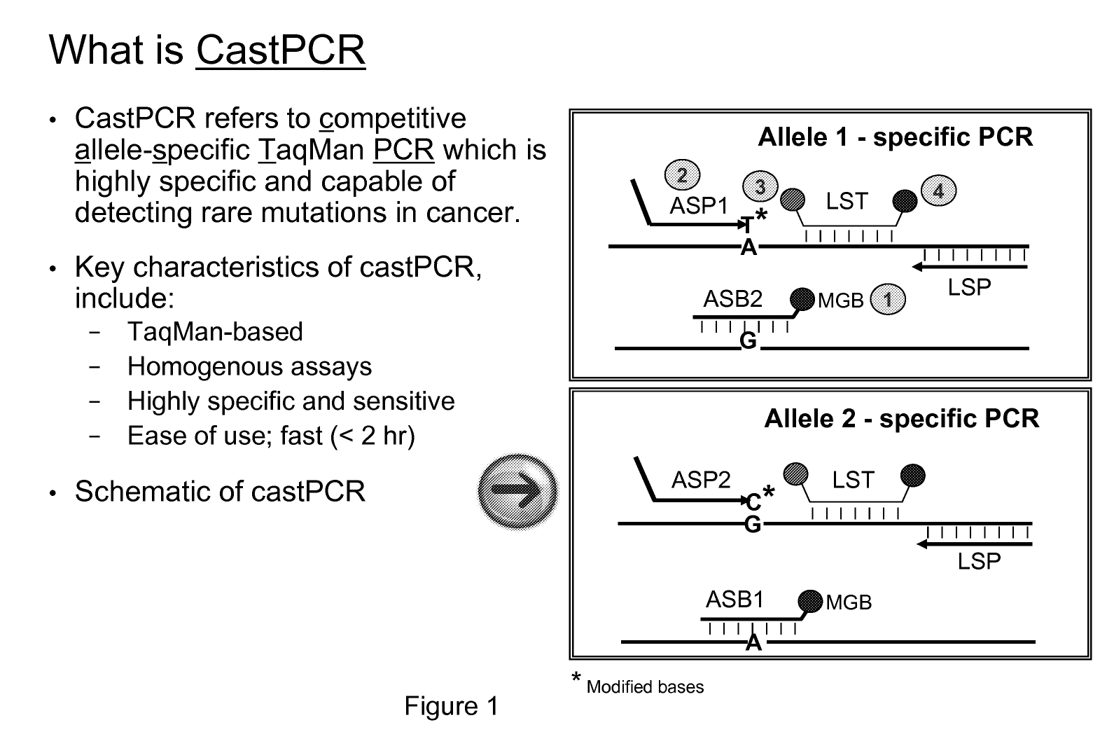

FIG. 2 demonstrates sensitivity and dynamic range of CastPCR.

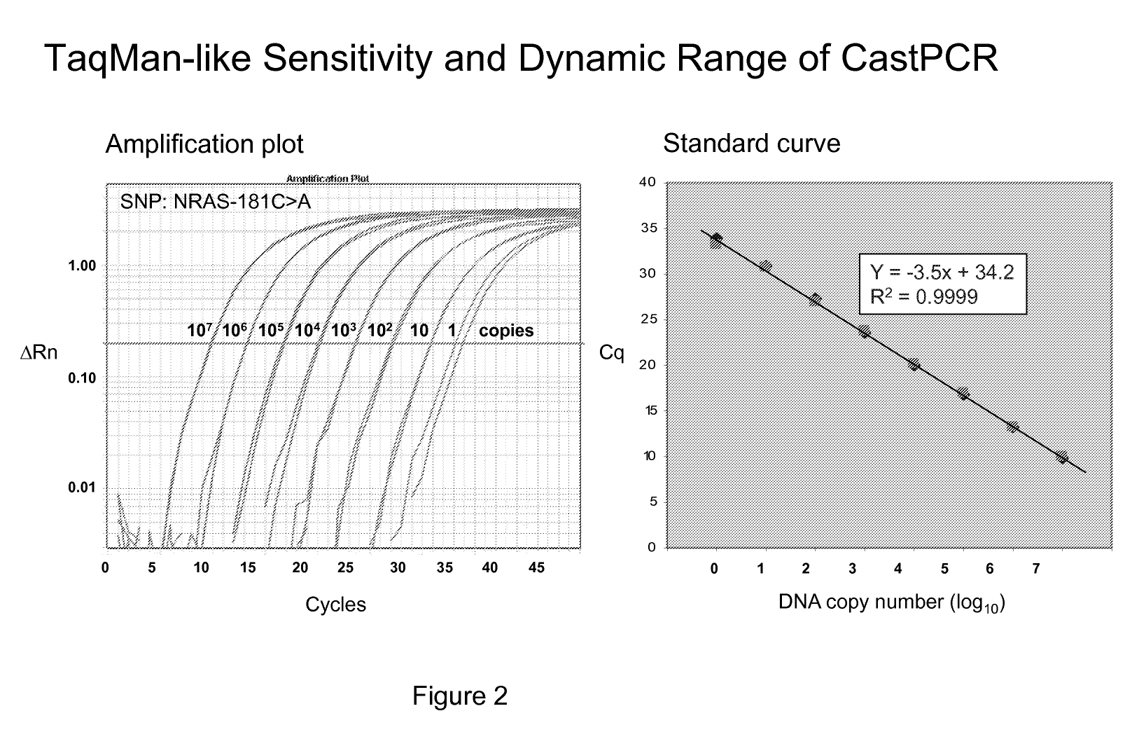

FIG. 3 shows comparison between AS-PCR and CastPCR.

FIG. 4 demonstrates specificity of CastPCR using synthetic DNA.

FIG. 5 demonstrates specificity of CastPCR using tumor genomic DNA spiking.

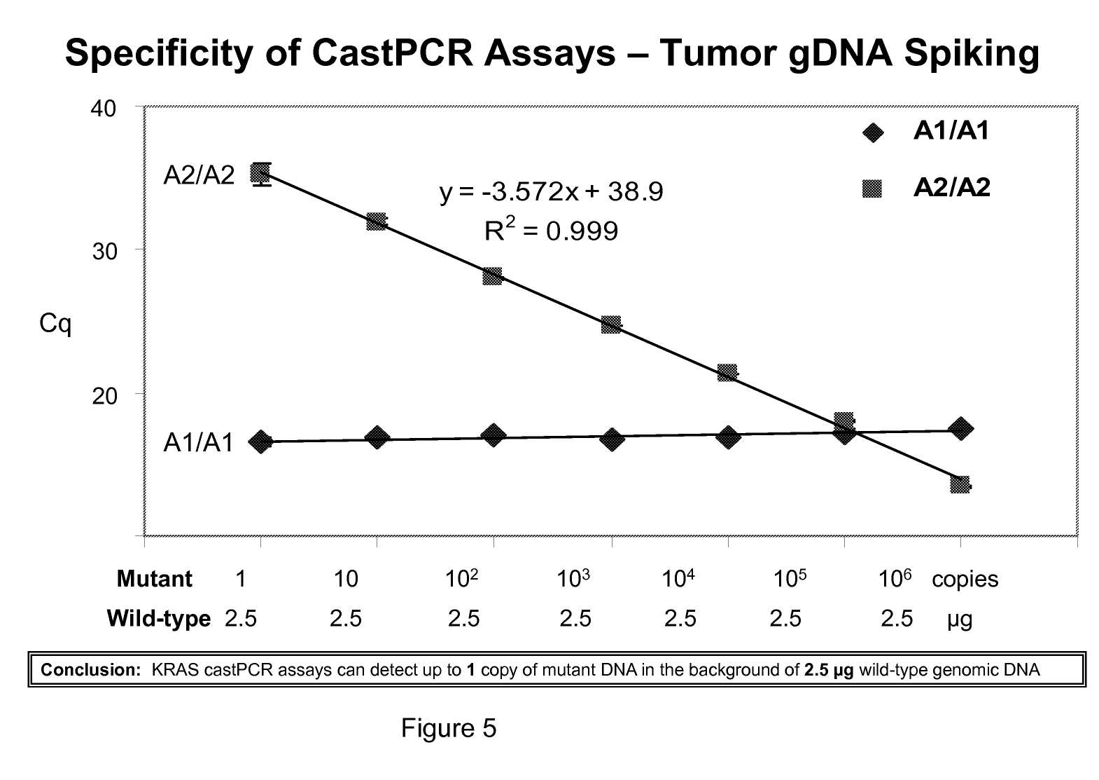

FIG. 6 shows a panel of cancer SNPs.

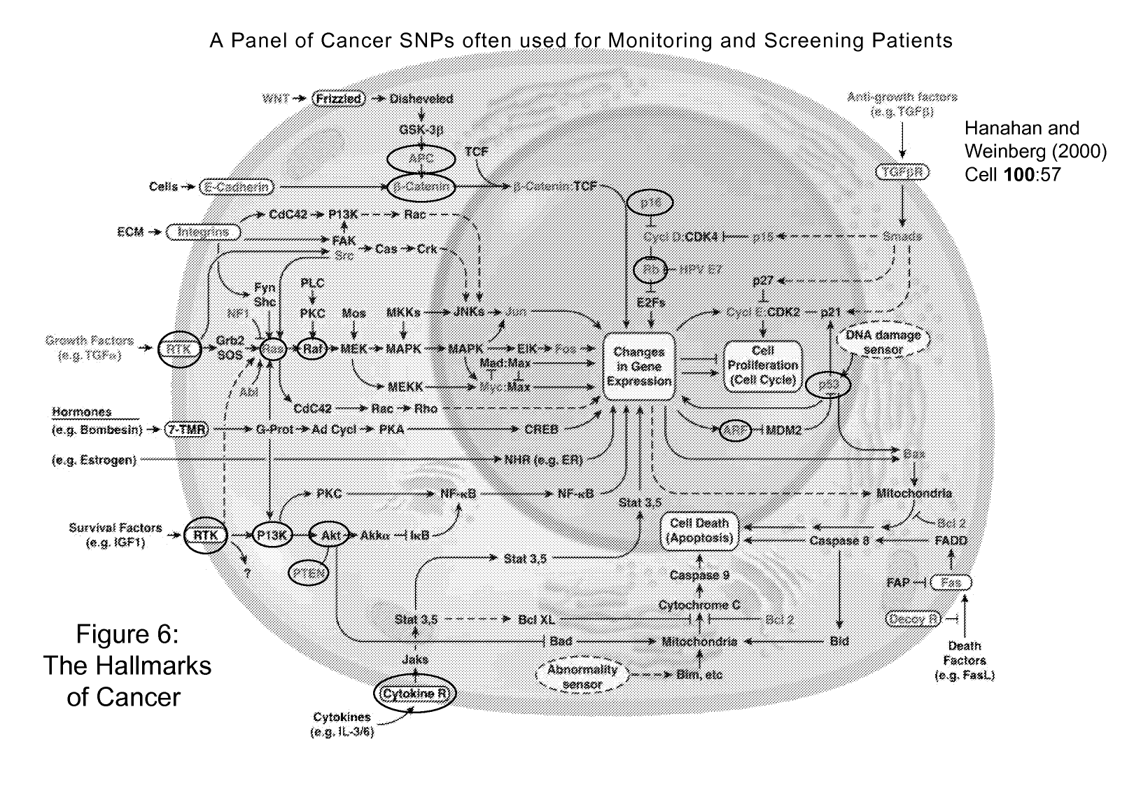

FIG. 7 is a schematic of multi-plex CastPCR.

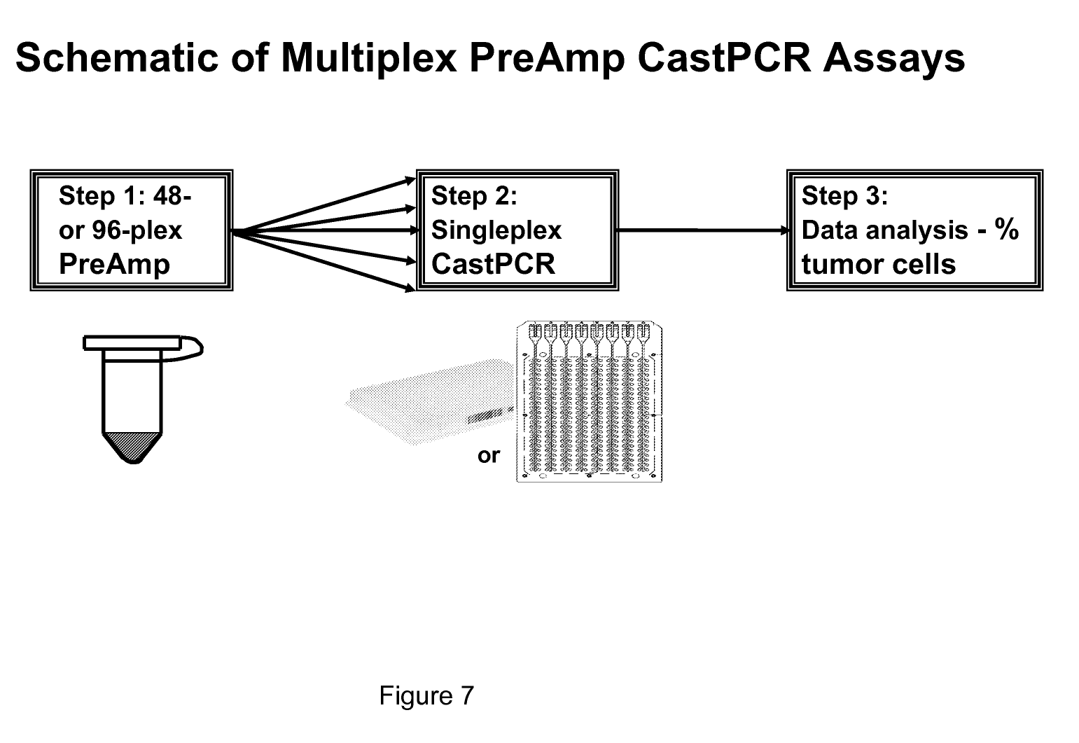

FIG. 8 summarizes CastPCR utility.

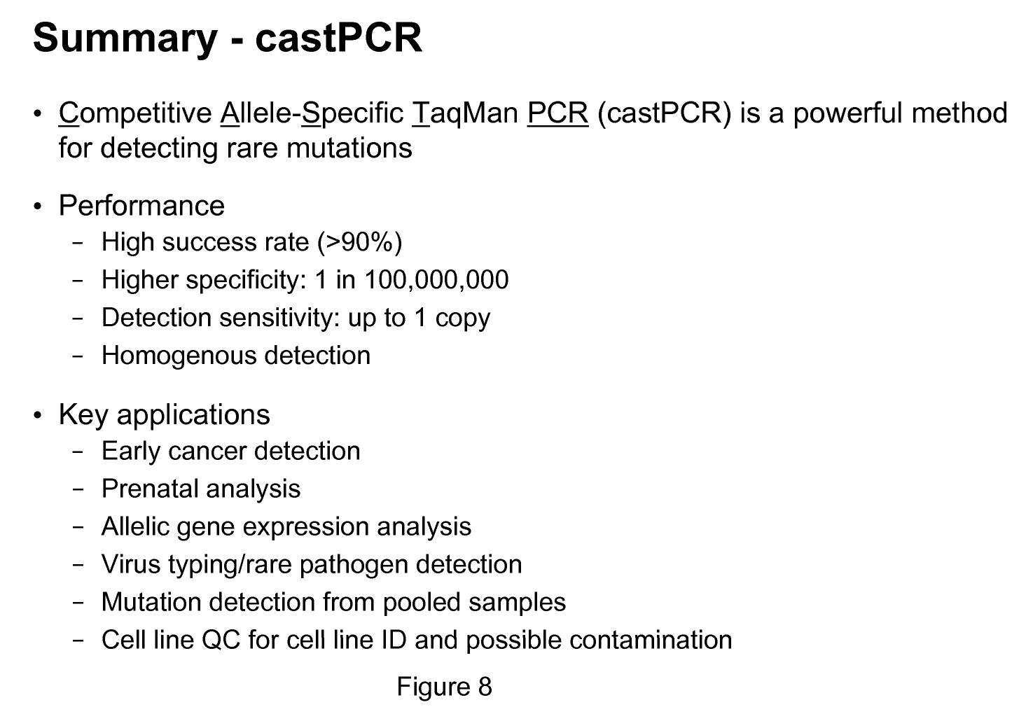

FIG. 9 describes the principle of methylation-specific CastPCR.

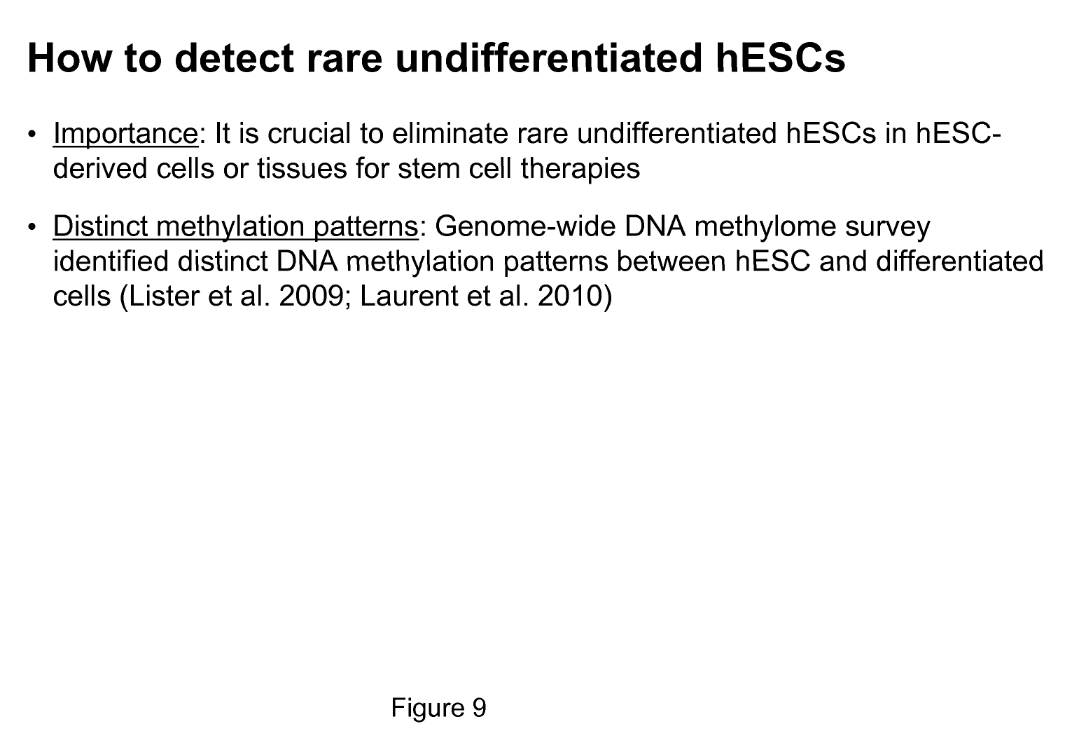

FIG. 10 is schematic of methylation-specific CastPCR.

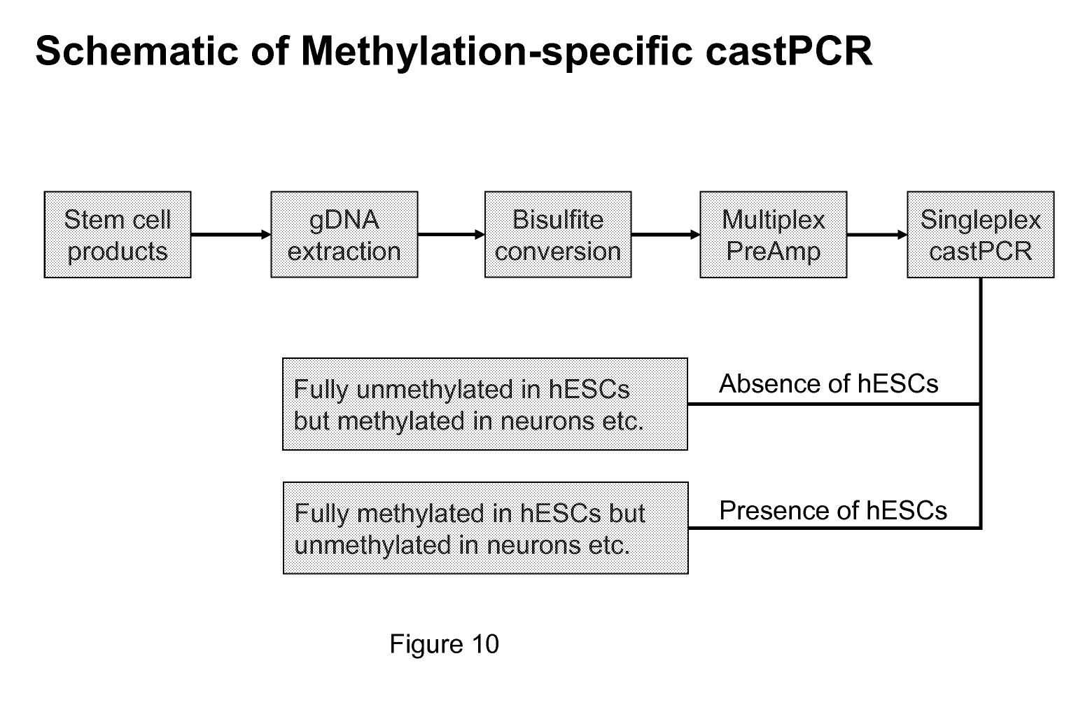

FIG. 11 is schematic of methylation-specific CastPCR in further detail.

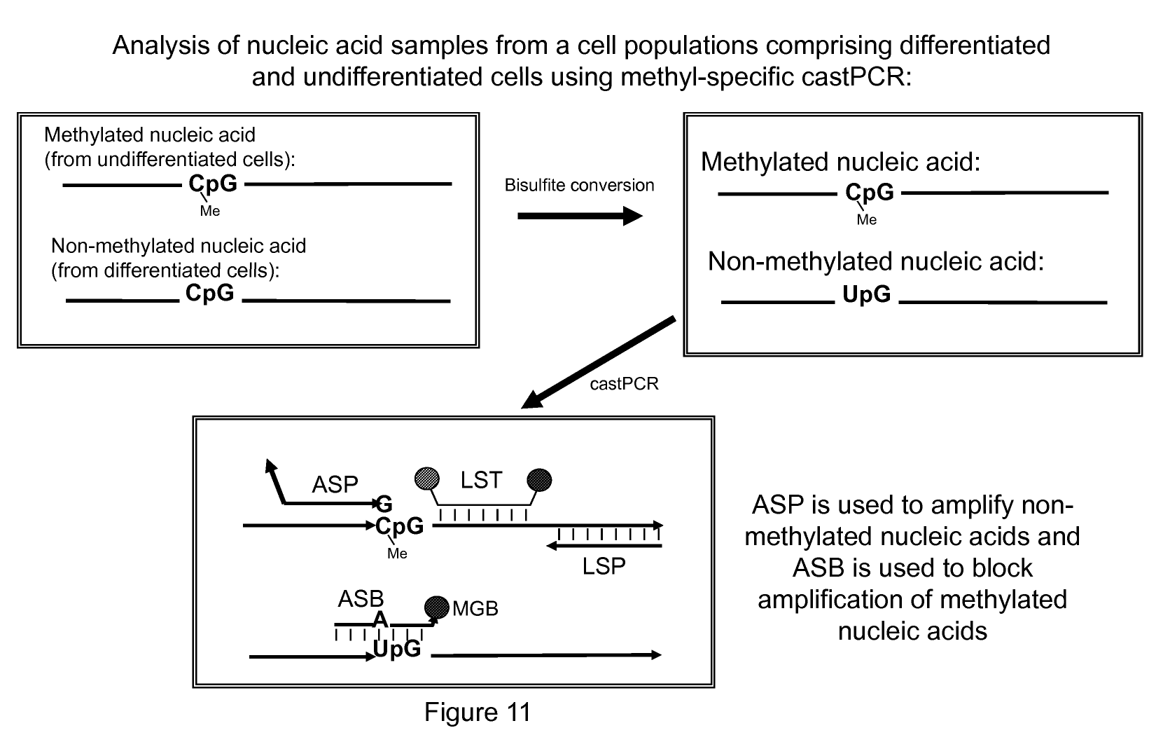

FIG. 12 summarizes methylation-specific CastPCR.

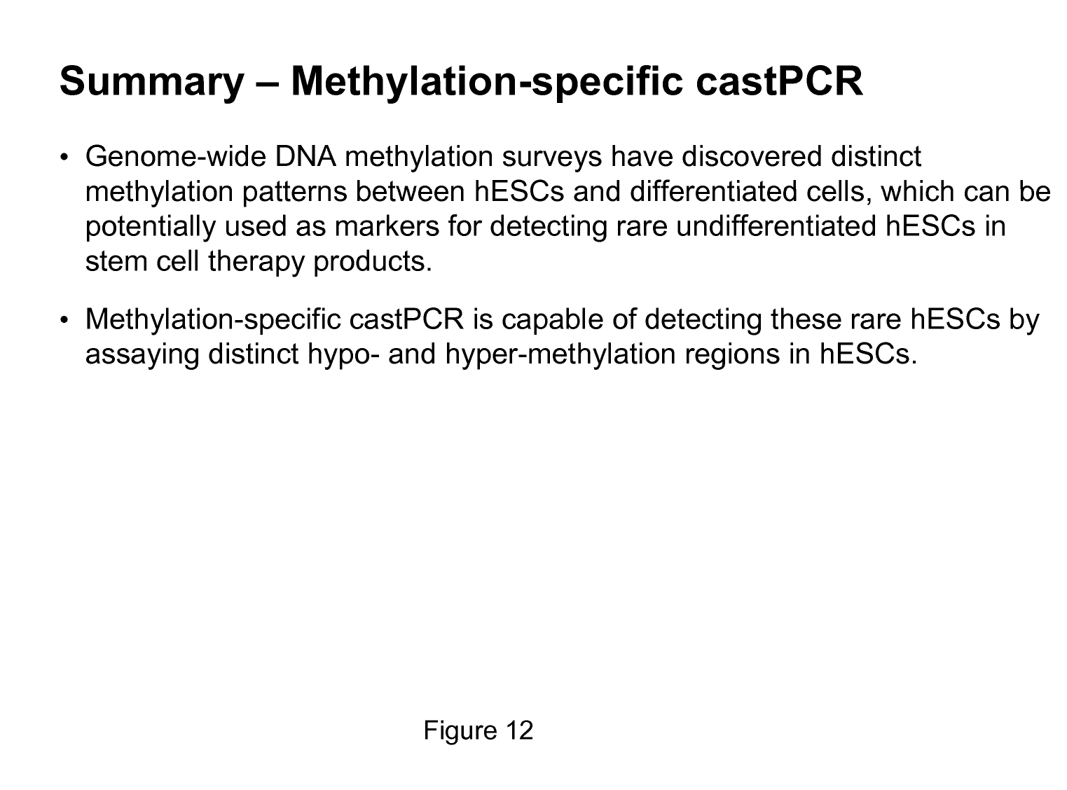

FIG. 13 is schematic of methylation-specific CastPCR in further detail to detect hESCs.

FIG. 14 demonstrates specificity of methylation-specific CastPCR.

FIG. 15 demonstrates sensitivity of methylation-specific CastPCR.

### DETAILED DESCRIPTION

### Introduction

CastPCR:

The selective amplification of an allele of interest is often complicated by factors including the mispriming and extension of a mismatched allele-specific primer on an alternative allele. Such mispriming and extension can be especially problematic in the detection of rare alleles present in a sample populated by an excess of another allelic variant. When in sufficient excess, the mispriming and extension of the other allelic variant may obscure the detection of the allele of interest. When using PCR-based methods, the discrimination of a particular allele in a sample containing alternative allelic variants relies on the selective amplification of an allele of interest, while minimizing or preventing amplification of other alleles present in the sample.

A number of factors have been identified, which alone or in combination, contribute to the enhanced discriminating power of allele-specific PCR. As disclosed herein, a factor which provides a greater ΔCt value between a mismatched and matched allele-specific primer is indicative of greater discriminating power between allelic variants. Such factors found to improve discrimination of allelic variants using the present methods include, for example, the use of one or more of the following: (a) tailed allele-specific primers; (b) low allele-specific primer concentration; (c) allele-specific primers designed to have lower Tm; (d) allele-specific primers designed to target discriminating bases; (e) allele-specific blocker probes designed to prevent amplification from alternative, and potentially more abundant, allelic variants in a sample; and (f) allele-specific blocker probes and/or allele-specific primers designed to comprise modified bases in order to increase the delta Tm between matched and mismatched target sequences.

The above-mentioned factors, especially when used in combination, can influence the ability of allele-specific PCR to discriminate between different alleles present in a sample. Thus, the present disclosure relates generally to novel amplification methods referred to as cast-PCR, which utilizes a combination of factors referred to above to improve discrimination of allelic variants during PCR by increasing ΔCt values. In some embodiments, the present methods can involve high levels of selectivity, wherein one mutant molecule in a background of at least 1,000 to 1,000,000, such as about 1000-10,000, about 10,000 to 100,000, or about 100,000 to 1,000,000 wild type molecules, or any fractional ranges in between can be detected. In some embodiments, the comparison of a first set of amplicons and a second set of amplicons involving the disclosed methods provides improvements in specificity from 1,000× to 100,000,000× fold difference, such as about 1000-10,000×, about 10,000 to 100,000×, about 100,000 to 1,000,000× or about 1,000,000 to 100,000,000× fold difference, or any fractional ranges in between.

Methylation: Most methods currently used to examine the DNA methylation patterns are biased toward CpG-rich regions of the genome, using methylation-sensitive restriction enzymes, affinity enrichment through methyl-cytosine-specific protein domains or antibodies, or bisulfite conversion. The classic method for single-base resolution of the cytosine methylation that occurs in mammalian DNA involves the use of sodium bisulfite to chemically convert non-methylated cytosines to uracils. After conversion, the DNA is amplified, typically by PCR, and in this process the uracils are re-encoded as thymidines. Sequences derived from this workflow are compared to reference (non-converted) sequence and C to T “mutations” are interpreted as representing cytosines that were non-methylated; conversely, cytosines that persist through this workflow are interpreted as having been methylated.

These studies have provided a snapshot of methylation status in a variety of cell types, but have low resolution and limited genomic coverage and are biased toward specific genomic features, such as CpG islands, promoter regions, or subsets of genes. Recent methodological improvements have revealed subtler methylation patterns that correlate with gene expression or cell type. For example, methylation at CpG sites located at the edges, or “shores,” of promoter-associated CpG islands has been inversely correlated with gene expression. Expressed protein-coding genes in general appear to have low methylation around their promoter region and high methylation over their gene body. In addition, comparison between human pluripotent stem cells and somatic cells revealed cell-type-specific areas of differential methylation.

The present disclosure takes advantage of known methylation status of certain genes that are differentially methylated in embryonic stem cells as compared to their differentiated progeny cells. By using the present disclosed invention, it is possible to detect a limited number of copies of unmethylated cytosine-containing sequences in presence of a large pool of methylated cytosine-containing sequences. This ability to distinguish methylated v. unmethylated sequences may be useful in various settings, for example, including but not limited to, embryonic stem cells v. their differentiated progeny cells; cancerous v. normal cells; fetal v. maternal cells; healthy v. infected cells. Evaluations of DNA methylation markers may also be useful as diagnostic indicators for early detection, risk assessment, therapeutic evaluation, recurrence monitoring for various diseases including cancers, AIDS, Alzheimer', and others.

### DEFINITIONS

For the purposes of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended.

As used herein, the term “allele” refers generally to alternative DNA sequences at the same physical locus on a segment of DNA, such as, for example, on homologous chromosomes. An allele can refer to DNA sequences which differ between the same physical locus found on homologous chromosomes within a single cell or organism or which differ at the same physical locus in multiple cells or organisms (“allelic variant”). In some instances, an allele can correspond to a single nucleotide difference at a particular physical locus. In other embodiments and allele can correspond to nucleotide (single or multiple) insertion or deletion.

As used herein, the term “allele-specific primer” refers to an oligonucleotide sequence that hybridizes to a sequence comprising an allele of interest, and which when used in PCR can be extended to effectuate first strand cDNA synthesis. Allele-specific primers are specific for a particular allele of a given target DNA or loci and can be designed to detect a difference of as little as one nucleotide in the target sequence. Allele-specific primers may comprise an allele-specific nucleotide portion, a target-specific portion, and/or a tail.

As used herein, the terms “allele-specific nucleotide portion” or “allele-specific target nucleotide” refers to a nucleotide or nucleotides in an allele-specific primer that can selectively hybridize and be extended from one allele (for example, a minor or mutant allele) at a given locus to the exclusion of the other (for example, the corresponding major or wild type allele) at the same locus.

As used herein, the term “target-specific portion” refers to the region of an allele-specific primer that hybridizes to a target polynucleotide sequence. In some embodiments, the target-specific portion of the allele-specific primer is the priming segment that is complementary to the target sequence at a priming region 5′ of the allelic variant to be detected. The target-specific portion of the allele-specific primer may comprise the allele-specific nucleotide portion. In other instances, the target-specific portion of the allele-specific primer is adjacent to the 3′ allele-specific nucleotide portion.

As used herein, the terms “tail” or “5′-tail” refers to the non-3′ end of a primer. This region typically will, although does not have to contain a sequence that is not complementary to the target polynucleotide sequence to be analyzed. The 5′ tail can be any of about 2-30, 2-5, 4-6, 5-8, 6-12, 7-15, 10-20, 15-25 or 20-30 nucleotides, or any range in between, in length.

As used herein, the term “allele-specific blocker probe” (also referred to herein as “blocker probe,” “blocker,”) refers to an oligonucleotide sequence that binds to a strand of DNA comprising a particular allelic variant which is located on the same, opposite or complementary strand as that bound by an allelic-specific primer, and reduces or prevents amplification of that particular allelic variant. As discussed in greater detail herein, allele-specific blocker probes generally comprise modifications, e.g., at the 3′-OH of the ribose ring, which prevent primer extension by a polymerase. The allele-specific blocker probe can be designed to anneal to the same or opposing strand of what the allele-specific primer anneals to and can be modified with a blocking group (e.g., a “non-extendable blocker moiety”) at its 3′ terminal end. Thus, a blocker probe can be designed, for example, so as to tightly bind to a wild type allele (e.g., abundant allelic variant) in order to suppress amplification of the wild type allele while amplification is allowed to occur on the same or opposing strand comprising a mutant allele (e.g., rare allelic variant) by extension of an allele-specific primer. In illustrative examples, the allele-specific blocker probes do not include a label, such as a fluorescent, radioactive, or chemiluminescent label.

As used herein, the term “non-extendable blocker moiety” refers generally to a modification on an oligonucleotide sequence such as a probe and/or primer which renders it incapable of extension by a polymerase, for example, when hybridized to its complementary sequence in a PCR reaction. Common examples of blocker moieties include modifications of the ribose ring 3′-OH of the oligonucleotide, which prevents addition of further bases to the ‘3-end of the oligonucleotide sequence a polymerase. Such 3′-OH modifications are well known in the art. (See, e.g., Josefsen, M., et al., Molecular and Cellular Probes, 23 (2009):201-223; McKinzie, P. et al., Mutagenesis. 2006, 21(6):391-7; Parsons, B. et al., Methods Mol Biol. 2005, 291:235-45; Parsons, B. et al., Nucleic Acids Res. 1992, 25:20(10):2493-6; and Morlan, J. et al., PLoS One 2009, 4 (2): e4584, the disclosures of which are incorporated herein by reference in their entireties.)

As used herein, the terms “MGB,” “MGB group,” “MGB compound,” or “MBG moiety” refers to a minor groove binder. When conjugated to the 3′ end of an oligonucleotide, an MGB group can function as a non-extendable blocker moiety.

An MGB is a molecule that binds within the minor groove of double stranded DNA. Although a general chemical formula for all known MGB compounds cannot be provided because such compounds have widely varying chemical structures, compounds which are capable of binding in the minor groove of DNA, generally speaking, have a crescent shape three dimensional structure. Most MGB moieties have a strong preference for A-T (adenine and thymine) rich regions of the B form of double stranded DNA. Nevertheless, MGB compounds which would show preference to C-G (cytosine and guanine) rich regions are also theoretically possible. Therefore, oligonucleotides comprising a radical or moiety derived from minor groove binder molecules having preference for C-G regions are also within the scope of the present invention.

Some MGBs are capable of binding within the minor groove of double stranded DNA with an association constant of 10³M⁻¹ or greater. This type of binding can be detected by well established spectrophotometric methods such as ultraviolet (UV) and nuclear magnetic resonance (NMR) spectroscopy and also by gel electrophoresis. Shifts in UV spectra upon binding of a minor groove binder molecule and NMR spectroscopy utilizing the “Nuclear Overhauser” (NOSEY) effect are particularly well known and useful techniques for this purpose. Gel electrophoresis detects binding of an MGB to double stranded DNA or fragment thereof, because upon such binding the mobility of the double stranded DNA changes.

A variety of suitable minor groove binders have been described in the literature. See, for example, Kutyavin, et al. U.S. Pat. No. 5,801,155; Wemmer, D. E., and Dervan P. B., Current Opinion in Structural Biology, 7:355-361 (1997); Walker, W. L., Kopka, J. L. and Goodsell, D. S., Biopolymers, 44:323-334 (1997); Zimmer, C.& Wahnert, U. Prog. Biophys. Molec. Bio. 47:31-112 (1986) and Reddy, B. S. P., Dondhi, S. M., and Lown, J. W., Pharmacol. Therap., 84:1-111 (1999) (the disclosures of which are herein incorporated by reference in their entireties). A preferred MGB in accordance with the present disclosure is DPI₃. Synthesis methods and/or sources for such MGBs are also well known in the art. (See, e.g., U.S. Pat. Nos. 5,801,155; 6,492,346; 6,084,102; and 6,727,356, the disclosures of which are incorporated herein by reference in their entireties.)

As used herein, the term “MGB blocker probe,” “MBG blocker,” or “MGB probe” is an oligonucleotide sequence and/or probe further attached to a minor groove binder moiety at its 3′ and/or 5′ end. Oligonucleotides conjugated to MGB moieties form extremely stable duplexes with single-stranded and double-stranded DNA targets, thus allowing shorter probes to be used for hybridization based assays. In comparison to unmodified DNA, MGB probes have higher melting temperatures (Tm) and increased specificity, especially when a mismatch is near the MGB region of the hybridized duplex. (See, e.g., Kutyavin, I. V., et al., Nucleic Acids Research, 2000, Vol. 28, No. 2: 655-661).

As used herein, the term “modified base” refers generally to any modification of a base or the chemical linkage of a base in a nucleic acid that differs in structure from that found in a naturally occurring nucleic acid. Such modifications can include changes in the chemical structures of bases or in the chemical linkage of a base in a nucleic acid, or in the backbone structure of the nucleic acid. (See, e.g., Latorra, D. et al., Hum Mut 2003, 2:79-85. Nakiandwe, J. et al., Plant Method 2007, 3:2.)

As used herein, the term “detector probe” refers to any of a variety of signaling molecules indicative of amplification. For example, SYBR® Green and other DNA-binding dyes are detector probes. Some detector probes can be sequence-based (also referred to herein as “locus-specific detector probe”), for example 5′ nuclease probes. Various detector probes are known in the art, for example (TaqMan® probes described herein (See also U.S. Pat. No. 5,538,848) various stem-loop molecular beacons (See, e.g., U.S. Pat. Nos. 6,103,476 and 5,925,517 and Tyagi and Kramer, 1996, Nature Biotechnology 14:303-308), stemless or linear beacons (See, e.g., WO 99/21881), PNA Molecular Beacons™ (See, e.g., U.S. Pat. Nos. 6,355,421 and 6,593,091), linear PNA beacons (See, e.g., Kubista et al., 2001, SPIE 4264:53-58), non-FRET probes (See, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161. Detector probes can comprise reporter dyes such as, for example, 6-carboxyfluorescein (6-FAM) or tetrachlorofluorescin (TET). Detector probes can also comprise quencher moieties such as tetramethylrhodamine (TAMRA), Black Hole Quenchers (Biosearch), Iowa Black (IDT), QSY quencher (Molecular Probes), and Dabsyl and Dabcel sulfonate/carboxylate Quenchers (Epoch). Detector probes can also comprise two probes, wherein for example a fluor is on one probe, and a quencher on the other, wherein hybridization of the two probes together on a target quenches the signal, or wherein hybridization on a target alters the signal signature via a change in fluorescence. Detector probes can also comprise sulfonate derivatives of fluorescein dyes with SO₃ instead of the carboxylate group, phosphoramidite forms of fluorescein, phosphoramidite forms of CY5 (available, for example, from Amersham Biosciences-GE Healthcare).

As used herein, the term “locus-specific primer” refers to an oligonucleotide sequence that hybridizes to products derived from the extension of a first primer (such as an allele-specific primer) in a PCR reaction, and which can effectuate second strand cDNA synthesis of said product. Accordingly, in some embodiments, the allele-specific primer serves as a forward PCR primer and the locus-specific primer serves as a reverse PCR primer, or vice versa. In some preferred embodiments, locus-specific primers are present at a higher concentration as compared to the allele-specific primers.

As used herein, the term “rare allelic variant” refers to a target polynucleotide present at a lower level in a sample as compared to an alternative allelic variant. The rare allelic variant may also be referred to as a “minor allelic variant” and/or a “mutant allelic variant.” For instance, the rare allelic variant may be found at a frequency less than 1/10, 1/100, 1/1,000, 1/10,000, 1/100,000, 1/1,000,000, 1/10,000,000, 1/100,000,000 or 1/1,000,000,000 compared to another allelic variant for a given SNP or gene. Alternatively, the rare allelic variant can be, for example, less than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, or 1,000,000 copies per 1, 10, 100, 1,000 micro liters of a sample or a reaction volume.

As used herein, the terms “abundant allelic variant” may refer to a target polynucleotide present at a higher level in a sample as compared to an alternative allelic variant. The abundant allelic variant may also be referred to as a “major allelic variant” and/or a “wild type allelic variant.” For instance, the abundant allelic variant may be found at a frequency greater than 10×, 100×, 1,000×, 10,000×, 100,000×, 1,000,000×, 10,000,000×, 100,000,000× or 1,000,000,000× compared to another allelic variant for a given SNP or gene. Alternatively, the abundant allelic variant can be, for example, greater than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, 1,000,000 copies per 1, 10, 100, 1,000 micro liters of a sample or a reaction volume.

As used herein, the terms “first” and “second” are used to distinguish the components of a first reaction (e.g., a “first” reaction; a “first” allele-specific primer) and a second reaction (e.g., a “second” reaction; a “second” allele-specific primer). By convention, as used herein the first reaction amplifies a first (for example, a rare) allelic variant and the second reaction amplifies a second (for example, an abundant) allelic variant or vice versa.

As used herein, both “first allelic variant” and “second allelic variant” can pertain to alleles of a given locus from the same organism. For example, as might be the case in human samples (e.g., cells) comprising wild type alleles, some of which have been mutated to form a minor or rare allele. The first and second allelic variants of the present teachings can also refer to alleles from different organisms. For example, the first allele can be an allele of a genetically modified organism, and the second allele can be the corresponding allele of a wild type organism. The first allelic variants and second allelic variants of the present teachings can be contained on gDNA, as well as mRNA and cDNA, and generally any target nucleic acids that exhibit sequence variability due to, for example, SNP or nucleotide(s) insertion and/or deletion mutations.

As used herein, the term “thermostable” or “thermostable polymerase” refers to an enzyme that is heat stable or heat resistant and catalyzes polymerization of deoxyribonucleotides to form primer extension products that are complementary to a nucleic acid strand. Thermostable DNA polymerases useful herein are not irreversibly inactivated when subjected to elevated temperatures for the time necessary to effect destabilization of single-stranded nucleic acids or denaturation of double-stranded nucleic acids during PCR amplification. Irreversible denaturation of the enzyme refers to substantial loss of enzyme activity. Preferably a thermostable DNA polymerase will not irreversibly denature at about 90°-100° C. under conditions such as is typically required for PCR amplification.

As used herein, the term “PCR amplifying” or “PCR amplification” refers generally to cycling polymerase-mediated exponential amplification of nucleic acids employing primers that hybridize to complementary strands, as described for example in Innis et al., PCR Protocols: A Guide to Methods and Applications, Academic Press (1990). Devices have been developed that can perform thermal cycling reactions with compositions containing fluorescent indicators which are able to emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; 6,174,670; and 6,814,934 and include, but are not limited to, the ABI Prism® 7700 Sequence Detection System (Applied Biosystems, Foster City, Calif.), the ABI GeneAmp® 5700 Sequence Detection System (Applied Biosystems, Foster City, Calif.), the ABI GeneAmp® 7300 Sequence Detection System (Applied Biosystems, Foster City, Calif.), the ABI GeneAmp® 7500 Sequence Detection System (Applied Biosystems, Foster City, Calif.), the StepOne™ Real-Time PCR System (Applied Biosystems, Foster City, Calif.) and the ABI GeneAmp® 7900 Sequence Detection System (Applied Biosystems, Foster City, Calif.).

As used herein, the term “Tm”’ or “melting temperature” of an oligonucleotide refers to the temperature (in degrees Celsius) at which 50% of the molecules in a population of a single-stranded oligonucleotide are hybridized to their complementary sequence and 50% of the molecules in the population are not-hybridized to said complementary sequence. The Tm of a primer or probe can be determined empirically by means of a melting curve. In some cases it can also be calculated using formulas well know in the art (See, e.g., Maniatis, T., et al., Molecular cloning: a laboratory manual/Cold Spring Harbor Laboratory, Cold Spring Harbor, N.Y.: 1982).

As used herein, the term “sensitivity” refers to the minimum amount (number of copies or mass) of a template that can be detected by a given assay. As used herein, the term “specificity” refers to the ability of an assay to distinguish between amplification from a matched template versus a mismatched template. Frequently, specificity is expressed as ΔCₜ=Ct_{mismatch}−Ct_{match}. An improvement in specificity or “specificity improvement” or “fold difference” is expressed herein as 2^{(ΔCt}^{_{—}}^{condition1-(ΔCt}^{_{—}}^{condition2)}. The term “selectivity” refers to the extent to which an AS-PCR assay can be used to determine minor (often mutant) alleles in mixtures without interferences from major (often wild type) alleles. Selectivity is often expressed as a ratio or percentage. For example, an assay that can detect 1 mutant template in the presence of 100 wild type templates is said to have a selectivity of 1:100 or 1%. As used herein, assay selectivity can also be calculated as ½^{Ct} or as a percentage using (½^{ΔCt}×100).

As used herein, the term “Ct” or “Ct value” refers to threshold cycle and signifies the cycle of a PCR amplification assay in which signal from a reporter that is indicative of amplicon generation (e.g., fluorescence) first becomes detectable above a background level. In some embodiments, the threshold cycle or “Ct” is the cycle number at which PCR amplification becomes exponential.

As used herein, the term “delta Ct” or “ΔCt” refers to the difference in the numerical cycle number at which the signal passes the fixed threshold between two different samples or reactions. In some embodiments delta Ct is the difference in numerical cycle number at which exponential amplification is reached between two different samples or reactions. The delta Ct can be used to identify the specificity between a matched primer to the corresponding target nucleic acid sequence and a mismatched primer to the same corresponding target nucleic acid sequence.

In some embodiments, the calculation of the delta Ct value between a mismatched primer and a matched primer is used as one measure of the discriminating power of allele-specific PCR. In general, any factor which increases the difference between the Ct value for an amplification reaction using a primer that is matched to a target sequence (e.g., a sequence comprising an allelic variant of interest) and that of a mismatched primer will result in greater allele discrimination power.

According to various embodiments, a Ct value may be determined using a derivative of a PCR curve. For example, a first, second, or nth order derivative method may be performed on a PCR curve in order to determine a Ct value. In various embodiments, a characteristic of a derivative may be used in the determination of a Ct value. Such characteristics may include, but are not limited by, a positive inflection of a second derivative, a negative inflection of a second derivative, a zero crossing of the second derivative, or a positive inflection of a first derivative. In various embodiments, a Ct value may be determined using a thresholding and baselining method. For example, an upper bound to an exponential phase of a PCR curve may be established using a derivative method, while a baseline for a PCR curve may be determined to establish a lower bound to an exponential phase of a PCR curve. From the upper and lower bound of a PCR curve, a threshold value may be established from which a Ct value is determined. Other methods for the determination of a Ct value known in the art, for example, but not limited by, various embodiments of a fit point method, and various embodiments of a sigmoidal method (See, e.g., U.S. Pat. Nos. 6,303,305; 6,503,720; 6,783,934, 7,228,237 and U.S. Application No. 2004/0096819; the disclosures of which are herein incorporated by reference in their entireties).

A nucleic acid may be methylated at one or more cytosines or adenines. Further, a nucleic acid may be hydroxymethylated on one or more cytosines. Also, a nucleic acid may be methylated on one or more guanines, uracils, or thymines and a nucleic acid may contain one or more of any of these modified bases. In some nucleic acids where methylation or hydroxymethylation is at the 5-carbon position of cytosine, non-methylated or non-hydroxymethylated cytosine may be deaminated while methylated cytosine remains unchanged. In some embodiments bisulfite may be used to deaminate the methylated or hydroxymethylated nucleic acid. In further embodiments, the nucleic acid contains one or more of the various known chemical modifications such as described in the texts Principles of Nucleic Acid Structure by W. Sanger (1984) and Nucleic Acids: Structures, Properties, and Functions by V. A. Bloomfield, D. M. Crothers, and I. Tinoco, Jr. (2000).

As used herein, the term “methylated”, when used in reference to nucleic acid, refers to nucleic acid which contains a methyl group on a base which is not normally present in nucleic acid when it is generated. In most cases, this base will be a cytosine and the methylated form will be 5-methylcytosine (“5-mC”). In some case, adenine may be methylated. The term methylated includes hemi-methylated and fully methylated nucleic acid.

As used herein, the term “hypermethylation” refers to the average methylation state corresponding to an increased presence of methylated bases (e.g., 5-mC) at one or a plurality of locations (e.g., CpG dinucleotides) within a nucleotide sequence, relative to the amount of methylated bases (e.g., 5-mC) found at corresponding location within a normal control nucleic acid sample. “Hypomethylation” is similar but relates to a decreased (vs. increased) presence of methylated bases.

### Compositions, Methods and Kits

In one aspect, the present invention provides compositions for use in identifying and/or quantitating an allelic variant in a nucleic acid sample. Some of these compositions can comprise: (a) an allele-specific primer; (b) an allele-specific blocker probe; (c) a detector probe; and (d) a locus-specific primer, or any combinations thereof. In some embodiments of the compositions, the compositions may further comprise a polymerase, dNTPs, reagents and/or buffers suitable for PCR amplification, and/or a template sequence or nucleic acid sample. In some embodiments, the polymerase can be thermostable.

In another aspect, the invention provides compositions comprising: (i) a first allele-specific primer, wherein an allele-specific nucleotide portion of the first allele-specific primer is complementary to the first allelic variant of a target sequence; and (ii) a first allele-specific blocker probe that is complementary to a region of the target sequence comprising the second allelic variant, wherein said region encompasses a position corresponding to the binding position of the allele-specific nucleotide portion of the first allele-specific primer, and wherein the first allele-specific blocker probe comprises a minor groove binder.

In some illustrative embodiments, the compositions can further include a locus-specific primer that is complementary to a region of the target sequence that is 3′ from the first allelic variant and on the opposite strand.

In further embodiments, the compositions can further include a detector probe.

In another aspect, the present invention provides methods for amplifying an allele-specific sequence. Some of these methods can include: (a) hybridizing an allele-specific primer to a first nucleic acid molecule comprising a target allele; (b) hybridizing an allele-specific blocker probe to a second nucleic acid molecule comprising an alternative allele wherein the alternative allele corresponds to the same loci as the target allele; (c) hybridizing a locus-specific detector probe to the first nucleic acid molecule; (d) hybridizing a locus-specific primer to the extension product of the allele-specific primer and (e) PCR amplifying the target allele.

In another aspect, the present invention provides methods for detecting and/or quantitating an allelic variant in a mixed sample. Some of these methods can involve: (a) in a first reaction mixture hybridizing a first allele-specific primer to a first nucleic acid molecule comprising a first allele (allele-1) and in a second reaction mixture hybridizing a second allele-specific primer to a first nucleic acid molecule comprising a second allele (allele-2), wherein the allele-2 corresponds to the same loci as allele-1; (b) in the first reaction mixture hybridizing a first allele-specific blocker probe to a second nucleic acid molecule comprising allele-2 and in the second reaction mixture hybridizing a second allele-specific blocker probe to a second nucleic acid molecule comprising allele-1; (c) in the first reaction mixture, hybridizing a first detector probe to the first nucleic acid molecule and in the second reaction mixture, hybridizing a second detector probe to the first nucleic acid molecule; (d) in the first reaction mixture hybridizing a first locus-specific primer to the extension product of the first allele-specific primer and in the second reaction mixture hybridizing a second locus-specific primer to the extension product of the second allele-specific primer; and (e) PCR amplifying the first nucleic acid molecule to form a first set or sample of amplicons and PCR amplifying the second nucleic acid molecule to form a second set or sample of amplicons; and (f) comparing the first set of amplicons to the second set of amplicons to quantitate allele-1 in the sample comprising allele-2 and/or allele-2 in the sample comprising allele-1.

In yet another aspect, the present invention provides methods for detecting and/or quantitating allelic variants. Some of these methods can comprise: (a) PCR amplifying a first allelic variant in a first reaction comprising (i) a low-concentration first allele-specific primer, (ii) a first locus-specific primer, and (iii) a first blocker probe to form first amplicons; (b) PCR amplifying a second allelic variant in a second reaction comprising (i) a low-concentration second allele-specific primer, (ii) a second locus-specific primer, and (iii) a second blocker probe to form second amplicons; and (d) comparing the first amplicons to the second amplicons to quantitate the first allelic variant in the sample comprising second allelic variants.

In yet another aspect, the present invention provides methods for detecting a first allelic variant of a target sequence in a nucleic acid sample suspected of comprising at least a second allelic variant of the target sequence. Methods of this aspect include forming a first reaction mixture by combining the following: (i) a nucleic acid sample; (ii) a first allele-specific primer, wherein an allele-specific nucleotide portion of the first allele-specific primer is complementary to the first allelic variant of the target sequence; (iii) a first allele-specific blocker probe that is complementary to a region of the target sequence comprising the second allelic variant, wherein said region encompasses a position corresponding to the binding position of the allele-specific nucleotide portion of the first allele-specific primer, and wherein the first allele-specific blocker probe comprises a minor groove binder; (iv) a first locus-specific primer that is complementary to a region of the target sequence that is 3′ from the first allelic variant and on the opposite strand; and (v) a first detector probe.

Next an amplification reaction, typically a PCR amplification reaction, is carried out on the first reaction mixture using the first locus-specific primer and the first allele-specific primer to form a first amplicon. Then, the first amplicon is detected by a change in a detectable property of the first detector probe upon binding to the amplicon, thereby detecting the first allelic variant of the target gene in the nucleic acid sample. The detector probe in some illustrative embodiments is a 5′ nuclease probe. The detectable property in certain illustrative embodiments is fluorescence.

In some embodiments, the 3′ nucleotide position of the 5′ target region of the first allele-specific primer is an allele-specific nucleotide position. In certain other illustrative embodiments, including those embodiments where the 3′ nucleotide position of the 5′ target region of the first allele-specific primer is an allele-specific nucleotide position, the blocking region of the allele-specific primer encompasses the allele-specific nucleotide position. Furthermore, in illustrative embodiments, the first allele-specific blocker probe includes a minor groove binder. Furthermore, the allele-specific blocker probe in certain illustrative embodiments does not have a label, for example a fluorescent label, or a quencher.

In certain illustrative embodiments, the quantity of the first allelic variant is determined by evaluating the change in a detectable property of the first detector probe.

In certain illustrative embodiments, the method further includes forming a second reaction mixture by combining (i) the nucleic acid sample; (ii) a second allele-specific primer, wherein an allele-specific nucleotide portion of the second allele-specific primer is complementary to the second allelic variant of the target sequence; (iii) a second allele-specific blocker probe that is complementary to a region of the target sequence comprising the first allelic variant, wherein said region encompasses a position corresponding to the binding position of the allele-specific nucleotide portion of the second allele-specific primer, and wherein the second allele-specific blocker probe comprises a minor groove binder; (iv) a second locus-specific primer that is complementary to a region of the target sequence that is 3′ from the second allelic variant and on the opposite strand; and (v) a second detector probe. Next, an amplification reaction is carried out on the second reaction mixture using the second allele-specific primer and the locus-specific primer, to form a second amplicon. Then the second amplicon is detected by a change in a detectable property of the detector probe.

In certain embodiments, the method further includes comparing the change in a detectable property of the first detector probe in the first reaction mixture to the change in a detectable property of the second detector probe in the second reaction mixture.

In preferred embodiments, the methods further include a 2-stage cycling protocol. In some embodiments, the cycling protocol comprises a first stage of amplification that employs an initial number of cycles at a lower annealing/extension temperature, followed by a second stage of amplification that employs a number of cycles at a higher annealing/extension temperature. Due to the lower Tm of cast-PCR allele-specific primers (e.g., 53-56° C.), PCR is not optimal at standard annealing/extension conditions (e.g., 60-64° C.). Consequently, lower annealing/extension temperatures are used during the initial cycling stage which improves cast-PCR efficiency significantly.

In some embodiments, the number of cycles used in the first stage of the cast-PCR cycling protocol is fewer than the number of cycles used in the second stage. In some embodiments of the cast-PCR methods, the number of cycles used in the first stage of the cycling protocol is about 2%-20%, 4%-18%, 6%-16%, 8%-14%, 10%-12%, or any percent in between, of the total number of cycles used in the second stage. In some embodiments, the first stage employs between 1 to 10 cycles, 2 to 8 cycles, 3 to 7 cycles, or 4 to 6 cycles, and all number of cycles in between, e.g., 2, 3, 4, 5, 6, or 7 cycles.

In some embodiments, the number of cycles used in the second stage of the cast-PCR cycling protocol is greater than the number of cycles used in the second stage. In some embodiments of the cast-PCR methods, the number of cycles used in the second stage of the cycling protocol is 5 times, 6 times, 8 times, 10 times, 12 times, 18 times, 25 times, or 30 times the number of cycles used in the first stage. In some embodiments, the second stage employs between 30 to 50 cycles, 35 to 48 cycles, 40 to 46 cycles, or any number of cycles in between, e.g., 42, 43, 44, 45, or 46 cycles.

In some embodiments, the lower annealing/extension temperature used during the first cycling stage is about 1° C., about 2° C., about 3° C., about 4° C., or about 5° C. lower than the annealing/extension temperature used during the second cycling stage. In some preferred embodiments, the annealing/extension temperature of the first stage is between 50° C. to 60° C., 52° C. to 58° C., or 54° C. to 56° C., e.g., 53° C., 54° C., 55° C. or 55° C. In some preferred embodiments the annealing/extension temperature of the second stage is between 56° C. to 66° C., 58° C. to 64° C., or 60° C. to 62° C., e.g., 58° C., 60° C., 62° C. or 64° C.

There are several major advantages of this 2-stage PCR cycling protocol used in cast-PCR that make it better than conventional AS-PCR methods. First, it improves the detection sensitivity by lowering the Ct value for matched targets or alleles. Next, it improves the specificity of cast-PCR by increasing the ΔCt between Ct values of matched and mismatched sequences. Finally, it can improve the uniformity of cast-PCR by making it equally efficient across various assays.

In yet another aspect, the present invention provides a reaction mixture that includes the following (i) nucleic acid molecule; (ii) an allele-specific primer, wherein an allele-specific nucleotide portion of the allele-specific primer is complementary to a first allelic variant of a target sequence; (iii) an allele-specific blocker probe that is complementary to a region of the target sequence comprising a second allelic variant, wherein said region encompasses a position corresponding to the binding position of the allele-specific nucleotide portion of the allele-specific primer, and wherein the allele-specific blocker probe comprises a minor groove binder; (iv) a locus-specific primer that is complementary to a region of the target sequence that is 3′ from the first allelic variant and on the opposite strand; and (v) a detector probe.

In certain embodiments, the methods of the invention are used to detect a first allelic variant that is present at a frequency less than 1/10, 1/100, 1/1,000, 1/10,000, 1/100,000, 1/1,000,000, 1/10,000,000, 1/100,000,000 or 1/1,000,000,000, and any fractional ranges in between, of a second allelic variant for a given SNP or gene. In other embodiments, the methods are used to detect a first allelic variant that is present in less than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, 1,000,000 copies per 1, 10, 100, 1,000 micro liters, and any fractional ranges in between, of a sample or a reaction volume.

In some embodiments, the first allelic variant is a mutant. In some embodiments the second allelic variant is wild type. In some embodiments, the present methods can involve detecting one mutant molecule in a background of at least 1,000 to 1,000,000, such as about 1000 to 10,000, about 10,000 to 100,000, or about 100,000 to 1,000,000 wild type molecules, or any fractional ranges in between. In some embodiments, the methods can provide high sensitivity and the efficiency at least comparable to that of TaqMan®-based assays.

In some embodiments, the comparison of the first amplicons and the second amplicons involving the disclosed methods provides improvements in specificity from 1,000× to 1,000,000× fold difference, such as about 1000 to 10,000×, about 10,000 to 100,000×, or about 100,000 to 1,000,000× fold difference, or any fractional ranges in between. In some embodiments, the size of the amplicons range from about 60-120 nucleotides long.

In another aspect, the present invention provides kits for quantitating a first allelic variant in a sample comprising an alternative second allelic variants that include: (a) a first allele-specific primer; (b) a second allele-specific primer; (c), a first locus-specific primer; (d) a second locus-specific primer; (e) a first allele-specific blocker probe; (f) a second allele-specific blocker probe; and (g) a polymerase. In some embodiments of the disclosed kits, the kit further comprises a first locus-specific detector probe and a second locus-specific detector probe.

In another aspect, the present invention provides kits that include two or more containers comprising the following components independently distributed in one of the two or more containers: (i) a first allele-specific primer, wherein an allele-specific nucleotide portion of the first allele-specific primer is complementary to the first allelic variant of a target sequence; and (ii) a first allele-specific blocker probe that is complementary to a region of the target sequence comprising the second allelic variant, wherein said region encompasses a position corresponding to the binding position of the allele-specific nucleotide portion of the first allele-specific primer, and wherein the first allele-specific blocker probe comprises a minor groove binder.

In some illustrative embodiments, the kits can further include a locus-specific primer that is complementary to a region of the target sequence that is 3′ from the first allelic variant and on the opposite strand.

In other embodiments, the kits can further include a detector probe.

In some embodiments, the compositions, methods, and/or kits can be used in detecting circulating cells in diagnosis. In one embodiment, the compositions, methods, and/or kits can be used to detect tumor cells in blood for early cancer diagnosis. In some embodiments, the compositions, methods, and/or kits can be used for cancer or disease-associated genetic variation or somatic mutation detection and validation. In some embodiments, the compositions, methods, and/or kits can be used for genotyping tetra-, tri- and di-allelic SNPs. In other embodiments, the compositions, methods, and/or kits can be used for identifying single or multiple nucleotide insertion or deletion mutations. In some embodiments, the compositions, methods, and/or kits can be used for DNA typing from mixed DNA samples for QC and human identification assays, cell line QC for cell contaminations, allelic gene expression analysis, virus typing/rare pathogen detection, mutation detection from pooled samples, detection of circulating tumor cells in blood, and/or prenatal diagnostics.

In some embodiments, the compositions, methods, and/or kits are compatible with various instruments such as, for example, SDS instruments from Applied Biosystems (Foster City, Calif.).

### Allele-Specific Primers

Allele-specific primers (ASPS) designed with low Tms exhibit increased discrimination of allelic variants. In some embodiments, the allele-specific primers are short oligomers ranging from about 15-30, such as about 16-28, about 17-26, about 18-24, or about 20-22, or any range in between, nucleotides in length. In some embodiments, the Tm of the allele-specific primers range from about 50° C. to 70° C., such as about 52° C. to 68° C., about 54° C. to 66° C., about 56° C. to 64° C., about 58° C. to 62° C., or any temperature in between (e.g., 53° C., 54° C., 55° C., 56° C.). In other embodiments, the Tm of the allele-specific primers is about 3° C. to 6° C. higher than the anneal/extend temperature of the PCR cycling conditions employed during amplification.

Low allele-specific primer concentration can also improve selectivity. Reduction in concentration of allele-specific primers below 900 nM can increase the delta Ct between matched and mismatched sequences. In some embodiments of the disclosed compositions, the concentration of allele-specific primers ranges from about 20 nM to 900 nM, such as about 50 nM to 700 nM, about 100 nM to 500 nM, about 200 nM to 300 nM, about 400 nM to 500 nM, or any range in between. In some exemplary embodiments, the concentration of the allele-specific primers is between about 200 nM to 400 nM.

In some embodiments, allele-specific primers can comprise an allele-specific nucleotide portion that is specific to the target allele of interest. The allele-specific nucleotide portion of an allele-specific primer is complementary to one allele of a gene, but not another allele of the gene. In other words, the allele-specific nucleotide portion binds to one or more variable nucleotide positions of a gene that is nucleotide positions that are known to include different nucleotides for different allelic variants of a gene. The allele-specific nucleotide portion is at least one nucleotide in length. In exemplary embodiments, the allele-specific nucleotide portion is one nucleotide in length. In some embodiments, the allele-specific nucleotide portion of an allele-specific primer is located at the 3′ terminus of the allele-specific primer. In other embodiments, the allele-specific nucleotide portion is located about 1-2, 3-4, 5-6, 7-8, 9-11, 12-15, or 16-20 nucleotides in from the 3′ most-end of the allele-specific primer.

Allele-specific primers designed to target discriminating bases can also improve discrimination of allelic variants. In some embodiments, the nucleotide of the allele-specific nucleotide portion targets a highly discriminating base (e.g., for detection of A/A, A/G, G/A, G/G, A/C, or C/A alleles). Less discriminating bases, for example, may involve detection of C/C, T/C, G/T, T/G, C/T alleles. In some embodiments, for example when the allele to be detected involves A/G or C/T SNPs, A or G may be used as the 3′ allele-specific nucleotide portion of the allele-specific primer (e.g., if A or T is the major allele), or C or T may be used as the 3′ allele-specific nucleotide portion of the allele-specific primer (e.g., if C or G is the major allele). In other embodiments, A may be used as the nucleotide-specific portion at the 3′ end of the allele specific primer (e.g., the allele-specific nucleotide portion) when detecting and/or quantifying A/T SNPs. In other embodiments, G may be used as the nucleotide-specific portion at the 3′ end of the allele specific primer when detecting and/or quantifying C/G SNPs.

In some embodiments, the allele-specific primer can comprise a target-specific portion that is specific to the polynucleotide sequence (or locus) of interest. In some embodiments the target-specific portion is about 75-85%, 85-95%, 95-99% or 100% complementary to the target polynucleotide sequence of interest. In some embodiments, the target-specific portion of the allele-specific primer can comprise the allele-specific nucleotide portion. In other embodiments, the target-specific portion is located 5′ to the allele-specific nucleotide portion. The target-specific portion can be about 4-30, about 5-25, about 6-20, about 7-15, or about 8-10 nucleotides in length. In some embodiments, the Tm of the target specific portion is about 5° C. below the anneal/extend temperature used for PCR cycling. In some embodiments, the Tm of the target specific portion of the allele-specific primer ranges from about 51° C. to 60° C., about 52° C. to 59° C., about 53° C. to 58° C., about 54° C. to 57° C., about 55° C. to 56° C., or about 50° C. to about 60° C.

In some embodiments of the disclosed methods and kits, the target-specific portion of the first allele-specific primer and the target-specific portion of the second allele-specific primer comprise the same sequence. In other embodiments, the target-specific portion of the first allele-specific primer and the target-specific portion of the second allele-specific primer are the same sequence.

In some embodiments, the allele-specific primer comprises a tail. Allele-specific primers comprising tails, enable the overall length of the primer to be reduced, thereby lowering the Tm without significant impact on assay sensitivity.

In some exemplary embodiments, the tail is on the 5′ terminus of the allele-specific primer. In some embodiments, the tail is located 5′ of the target-specific portion and/or allele-specific nucleotide portion of the allele-specific primer. In some embodiments, the tail is about 65-75%, about 75-85%, about 85-95%, about 95-99% or about 100% non-complementary to the target polynucleotide sequence of interest. In some embodiments the tail can be about 2-40, such as about 4-30, about 5-25, about 6-20, about 7-15, or about 8-10 nucleotides in length. In some embodiments the tail is GC-rich. For example, in some embodiments the tail sequence is comprised of about 50-100%, about 60-100%, about 70-100%, about 80-100%, about 90-100% or about 95-100% G and/or C nucleotides.

The tail of the allele-specific primer may be configured in a number of different ways, including, but not limited to a configuration whereby the tail region is available after primer extension to hybridize to a complementary sequence (if present) in a primer extension product. Thus, for example, the tail of the allele-specific primer can hybridize to the complementary sequence in an extension product resulting from extension of a locus-specific primer.

In some embodiments of the disclosed methods and kits, the tail of the first allele-specific primer and the tail of the second allele-specific primer comprise the same sequence. In other embodiments, the 5′ tail of the first allele-specific primer and the 5′ tail of the second allele-specific primer are the same sequence.

### Allele-Specific Blocker Probes

Allele-specific blocker probes (or ASBs) (herein sometimes referred to as “blocker probes”) may be designed as short oligomers that are single-stranded and have a length of 100 nucleotides or less, more preferably 50 nucleotides or less, still more preferably 30 nucleotides or less and most preferably 20 nucleotides or less with a lower limit being approximately 5 nucleotides.

In some embodiments, the Tm of the blocker probes range from 58° C. to 70° C., 61° C. to 69° C., 62° C. to 68° C., 63° C. to 67° C., 64° C. to 66° C., or about 60° C. to about 63° C., or any range in between. In yet other embodiments, the Tm of the allele-specific blocker probes is about 3° C. to 6° C. higher than the anneal/extend temperature in the PCR cycling conditions employed during amplification.

In some embodiments, the blocker probes are not cleaved during PCR amplification. In some embodiments, the blocker probes comprise a non-extendable blocker moiety at their 3′-ends. In some embodiments, the blocker probes can further comprise other moieties (including, but not limited to additional non-extendable blocker moieties, quencher moieties, fluorescent moieties, etc) at their 3′-end, 5′-end, and/or any internal position in between. In some embodiments, the allele position is located about 5-15, such as about 5-11, about 6-10, about 7-9, about 7-12, or about 9-11, such as about 6, about 7, about 8, about 9, about 10, or about 11 nucleotides away from the non-extendable blocker moiety of the allele-specific blocker probes when hybridized to their target sequences. In some embodiments, the non-extendable blocker moiety can be, but is not limited to, an amine (NH₂), biotin, PEG, DPI₃, or PO₄. In some preferred embodiments, the blocker moiety is a minor groove binder (MGB) moiety. (The oligonucleotide-MGB conjugates of the present invention are hereinafter sometimes referred to as “MGB blocker probes” or “MGB blockers.”)

As disclosed herein, the use of MGB moieties in allele-specific blocker probes can increase the specificity of allele-specific PCR. One possibility for this effect is that, due to their strong affinity to hybridize and strongly bind to complementary sequences of single or double stranded nucleic acids, MGBs can lower the Tm of linked oligonucleotides (See, for example, Kutyavin, I., et al., Nucleic Acids Res., 2000, Vol. 28, No. 2: 655-661). Oligonucleotides comprising MGB moieties have strict geometric requirements since the linker between the oligonucleotide and the MGB moiety must be flexible enough to allow positioning of the MGB in the minor groove after DNA duplex formation. Thus, MGB blocker probes can provide larger Tm differences between matched versus mismatched alleles as compared to conventional DNA blocker probes.

In general, MGB moieties are molecules that bind within the minor groove of double stranded DNA. Although a generic chemical formula for all known MGB compounds cannot be provided because such compounds have widely varying chemical structures, compounds which are capable of binding in the minor groove of DNA, generally speaking, have a crescent shape three dimensional structure. Most MGB moieties have a strong preference for A-T (adenine and thymine) rich regions of the B form of double stranded DNA. Nevertheless, MGB compounds which would show preference to C-G (cytosine and guanine) rich regions are also theoretically possible. Therefore, oligonucleotides comprising a radical or moiety derived from minor groove binder molecules having preference for C-G regions are also within the scope of the present invention.

Some MGBs are capable of binding within the minor groove of double stranded DNA with an association constant of 10³M⁻¹ or greater. This type of binding can be detected by well established spectrophotometric methods such as ultraviolet (UV) and nuclear magnetic resonance (NMR) spectroscopy and also by gel electrophoresis. Shifts in UV spectra upon binding of a minor groove binder molecule and NMR spectroscopy utilizing the “Nuclear Overhauser” (NOSEY) effect are particularly well known and useful techniques for this purpose. Gel electrophoresis detects binding of an MGB to double stranded DNA or fragment thereof, because upon such binding the mobility of the double stranded DNA changes.

A variety of suitable minor groove binders have been described in the literature. See, for example, Kutyavin, et al. U.S. Pat. No. 5,801,155; Wemmer, D. E., and Dervan P. B., Current Opinion in Structural Biology, 7:355-361 (1997); Walker, W. L., Kopka, J. L. and Goodsell, D. S., Biopolymers, 44:323-334 (1997); Zimmer, C.& Wahnert, U. Prog. Biophys. Molec. Bio. 47:31-112 (1986) and Reddy, B. S. P., Dondhi, S. M., and Lown, J. W., Pharmacol. Therap., 84:1-111 (1999). In one group of embodiments, the MGB is selected from the group consisting of CC1065 analogs, lexitropsins, distamycin, netropsin, berenil, duocarmycin, pentamidine, 4,6-diamino-2-phenylindole and pyrrolo[2,1-c][1,4]benzodiazepines. A preferred MGB in accordance with the present disclosure is DPI₃ (see U.S. Pat. No. 6,727,356, the disclosure of which is incorporated herein by reference in its entirety).

Suitable methods for attaching MGBs through linkers to oligonucleotides or probes and have been described in, for example, U.S. Pat. Nos. 5,512,677; 5,419,966; 5,696,251; 5,585,481; 5,942,610; 5,736,626; 5,801,155 and 6,727,356. (The disclosures of each of which are incorporated herein by reference in their entireties.) For example, MGB-oligonucleotide conjugates can be synthesized using automated oligonucleotide synthesis methods from solid supports having cleavable linkers. In other examples, MGB probes can be prepared from an MGB modified solid support substantially in accordance with the procedure of Lukhtanov et al. Bioconjugate Chem., 7: 564-567 (1996). (The disclosure of which is also incorporated herein by reference in its entirety.) According to these methods, one or more MGB moieties can be attached at the 5′-end, the 3′-end and/or at any internal portion of the oligonucleotide.

The location of an MGB moiety within an MGB-oligonucleotide conjugate can affect the discriminatory properties of such a conjugate. An unpaired region within a duplex will likely result in changes in the shape of the minor groove in the vicinity of the mismatched base(s). Since MGBs fit best within the minor groove of a perfectly-matched DNA duplex, mismatches resulting in shape changes in the minor groove would reduce binding strength of an MGB to a region containing a mismatch. Hence, the ability of an MGB to stabilize such a hybrid would be decreased, thereby increasing the ability of an MGB-oligonucleotide conjugate to discriminate a mismatch from a perfectly-matched duplex. On the other hand, if a mismatch lies outside of the region complementary to an MGB-oligonucleotide conjugate, discriminatory ability for unconjugated and MGB-conjugated oligonucleotides of equal length is expected to be approximately the same. Since the ability of an oligonucleotide probe to discriminate single base pair mismatches depends on its length, shorter oligonucleotides are more effective in discriminating mismatches. The first advantage of the use of MGB-oligonucleotides conjugates in this context lies in the fact that much shorter oligonucleotides compared to those used in the art (i.e., 20-mers or shorter), having greater discriminatory powers, can be used, due to the pronounced stabilizing effect of MGB conjugation. Consequently, larger delta Tms of allele-specific blocker probes can improve AS-PCR assay specificity and selectivity.

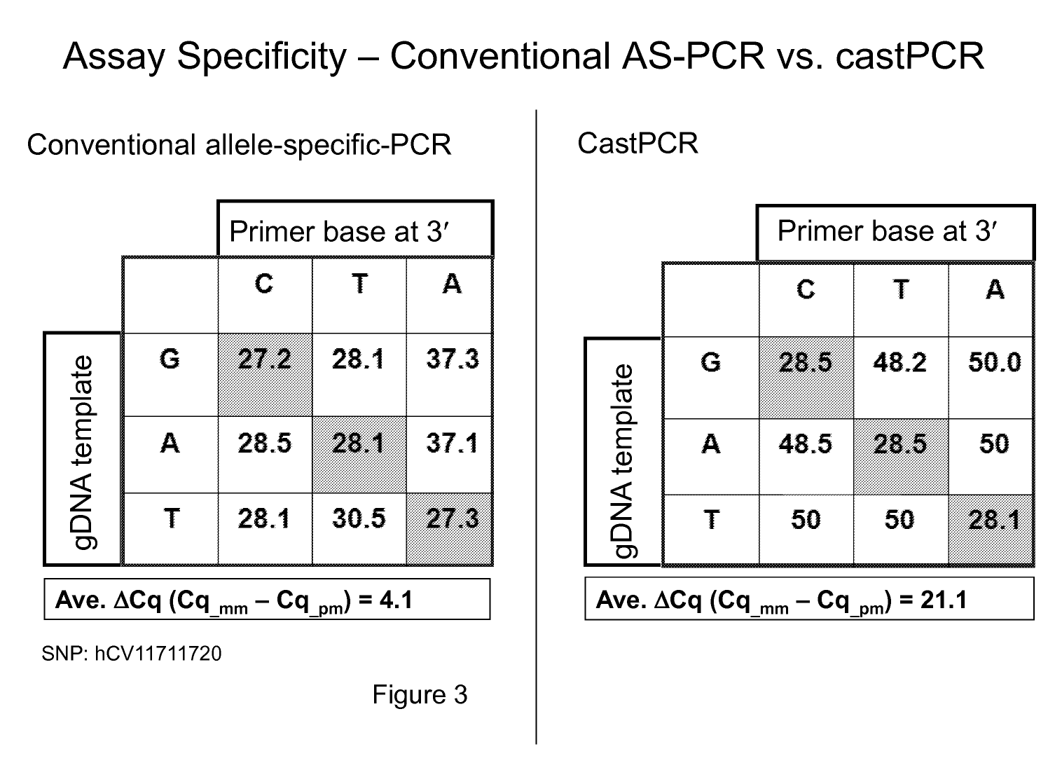

Blocker probes having MGB at the 5′ termini may have additional advantages over other blocker probes having a blocker moiety (e.g., MGB, PO₄, NH₂, PEG, or biotin) only at the 3′ terminus. This is at least because blocker probes having MGB at the 5′ terminus (in addition to a blocking moiety at the 3′-end that prevents extension) will not be cleaved during PCR amplification. Thus, the probe concentration can be maintained at a constant level throughout PCR, which may help maintain the effectiveness of blocking non-specific priming, thereby increasing cast-PCR assay specificity and selectivity (FIG. 3).

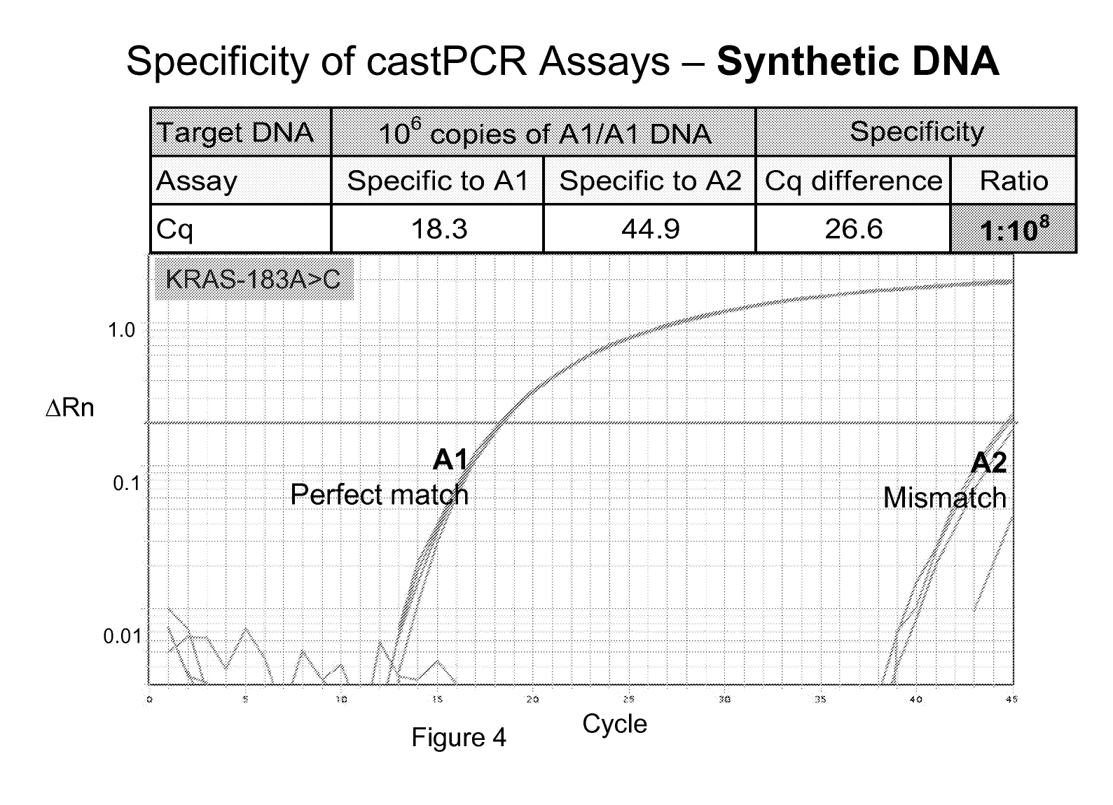

In some embodiments, as depicted in FIG. 4A, the allele-specific primer and/or the allele-specific blocker probe can comprise one or more modified nucleobases or nucleosidic bases different from the naturally occurring bases (i.e., adenine, cytosine, guanine, thymine and uracil). In some embodiments, the modified bases are still able to effectively hybridize to nucleic acid units that contain adenine, guanine, cytosine, uracil or thymine moieties. In some embodiments, the modified base(s) may increase the difference in the Tm between matched and mismatched target sequences and/or decrease mismatch priming efficiency, thereby improving not only assay specificity, bust also selectivity.

Modified bases are considered to be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. In some embodiments, all tautomeric forms of naturally occurring bases, modified bases and base analogues may also be included in the oligonucleotide primers and probes of the invention.

Some examples of modified base(s) may include, for example, the general class of base analogues 7-deazapurines and their derivatives and pyrazolopyrimidines and their derivatives (described in PCT WO 90/14353; and U.S. application Ser. No. 09/054,630, the disclosures of each of which are incorporated herein by reference in their entireties). Examples of base analogues of this type include, for example, the guanine analogue 6-amino-1H-pyrazolo[3,4-d]pyrimidin-4(5H)-one (ppG), the adenine analogue 4-amino-1H-pyrazolo[3,4-d]pyrimidine (ppA), and the xanthine analogue 1H-pyrazolo[4,4-d]pyrimidin-4(5H)-6(7H)-dione (ppX). These base analogues, when present in an oligonucleotide of some embodiments of this invention, strengthen hybridization and can improve mismatch discrimination.

Additionally, in some embodiments, modified sugars or sugar analogues can be present in one or more of the nucleotide subunits of an oligonucleotide conjugate in accordance with the invention. Sugar modifications include, but are not limited to, attachment of substituents to the 2′, 3′ and/or 4′ carbon atom of the sugar, different epimeric forms of the sugar, differences in the α- or β-configuration of the glycosidic bond, and other anomeric changes. Sugar moieties include, but are not limited to, pentose, deoxypentose, hexose, deoxyhexose, ribose, deoxyribose, glucose, arabinose, pentofuranose, xylose, lyxose, and cyclopentyl.

Locked nucleic acid (LNA)-type modifications, for example, typically involve alterations to the pentose sugar of ribo- and deoxyribonucleotides that constrains, or “locks,” the sugar in the N-type conformation seen in A-form DNA. In some embodiments, this lock can be achieved via a 2′-O,4′-C methylene linkage in 1,2:5,6-di-O-isopropylene-α-D-allofuranose. In other embodiments, this alteration then serves as the foundation for synthesizing locked nucleotide phosphoramidite monomers. (See, for example, Wengel J., Acc. Chem. Res., 32:301-310 (1998), U.S. Pat. No. 7,060,809; Obika, et al., Tetrahedron Lett 39: 5401-5405 (1998); Singh, et al., Chem Commun 4:455-456 (1998); Koshkin, et al., Tetrahedron 54: 3607-3630 (1998), the disclosures of each of which are incorporated herein by reference in their entireties.)

In some preferred embodiments, the modified bases include 8-Aza-7-deaza-dA (ppA), 8-Aza-7-deaza-dG (ppG), 2′-Deoxypseudoisocytosine (iso dC), 5-fluoro-2′-deoxyuracil (fdU), locked nucleic acid (LNA), or 2′-O,4′-C-ethylene bridged nucleic acid (ENA) bases. Other examples of modified bases that can be used in the invention are depicted in FIG. 4B and described in U.S. Pat. No. 7,517,978 (the disclosure of which is incorporated herein by reference in its entirety).

Many modified bases, including for example, LNA, ppA, ppG, 5-Fluoro-dU (fdU), are commercially available and can be used in oligonucleotide synthesis methods well known in the art. In some embodiments, synthesis of modified primers and probes can be carried out using standard chemical means also well known in the art. For example, in certain embodiments, the modified moiety or base can be introduced by use of a (a) modified nucleoside as a DNA synthesis support, (b) modified nucleoside as a phosphoramidite, (c) reagent during DNA synthesis (e.g., benzylamine treatment of a convertible amidite when incorporated into a DNA sequence), or (d) by post-synthetic modification.

In some embodiments, the primers or probes are synthesized so that the modified bases are positioned at the 3′ end. In some embodiments, the modified base are located between, 1-6 nucleotides, e.g., 2, 3, 4 or 5 nucleotides away from the 3′-end of the allele-specific primer or blocker probe. In some preferred embodiments, the primers or probes are synthesized so that the modified bases are positioned at the 3′-most end of the allele-specific primer or blocker probe.

Modified internucleotide linkages can also be present in oligonucleotide conjugates of the invention. Such modified linkages include, but are not limited to, peptide, phosphate, phosphodiester, phosphotriester, alkylphosphate, alkanephosphonate, thiophosphate, phosphorothioate, phosphorodithioate, methylphosphonate, phosphoramidate, substituted phosphoramidate and the like. Several further modifications of bases, sugars and/or internucleotide linkages, that are compatible with their use in oligonucleotides serving as probes and/or primers, will be apparent to those of skill in the art.

In addition, in some embodiments, the nucleotide units which are incorporated into the oligonucleotides of the allele-specific primers and/or blocker probes of the present invention may have a cross-linking function (an alkylating agent) covalently bound to one or more of the bases, through a linking arm.

In some embodiments of the methods and kits, the first allele-specific blocker probe binds to the same strand or sequence as the first allele-specific primer, while the second allele-specific blocker probe binds to the opposite strand and/or complementary sequence as the first allele-specific primer.

### Detector Probes

In some embodiments, detector probe is designed as short oligomers ranging from about 15-30 nucleotides, such as about 16, about 18, about 22, about 24, about 30, or any number in between. In some embodiments, the Tm of the detector probe ranges from about 60° C. to 80° C., about 61° C. to 69° C., about 62° C. to 68° C., about 63° C. to 67° C., or about 64° C. to 66° C., or any range in between.

In some embodiments, the detector probe is a locus-specific detector probes (LST). In other embodiments the detector probe is a 5′ nuclease probe. In some exemplary embodiments, the detector probe can comprises an MGB moiety, a reporter moiety (e.g., FAM™, TEr™, JOE™, VIC™, or SYBR® Green), a quencher moiety (e.g., Black Hole Quencher™ or TAMRA™), and/or a passive reference (e.g., ROX™). In some exemplary embodiments, the detector probe is designed according to the methods and principles described in U.S. Pat. No. 6,727,356 (the disclosure of which is incorporated herein by reference in its entirety). In some exemplary embodiments, the detector probe is a TaqMan® probe (Applied Biosystems, Foster City). In exemplary embodiments, the locus-specific detector probe can be designed according to the principles and methods described in U.S. Pat. No. 6,727,356 (the disclosure of which is incorporated herein by reference in its entirety). For example, fluorogenic probes can be prepared with a quencher at the 3′ terminus of a single DNA strand and a fluorophore at the 5′ terminus. In such an example, the 5′-nuclease activity of a Taq DNA polymerase can cleave the DNA strand, thereby separating the fluorophore from the quencher and releasing the fluorescent signal. In some embodiments, the detector probes are hybridized to the template strands during primer extension step of PCR amplification (e.g., at 60-65° C.). In yet other embodiments, an MGB is covalently attached to the quencher moiety of the locus-specific detector probes (e.g., through a linker).

In some embodiments of the disclosed methods and kits, the first and second detector probes are the same and/or comprise the same sequence or are the same sequence.

### Locus-Specific Primers

In some embodiments, locus-specific primer (LSP) is designed as a short oligomer ranging from about 15-30 nucleotides, such as about 16, about 18, about 22, about 24, about 30, or any number in between. In some embodiments, the Tm of the locus-specific primer ranges from about 60° C. to 70° C., about 61° C. to 69° C., about 62° C. to 68° C., about 63° C. to 67° C., or about 64° C. to 66° C., or any range in between.

In some other embodiments of the disclosed methods and kits, the first locus-specific detector probe and/or second locus-specific detector probes comprise the same sequence or are the same sequence.

### Additional Components

Polymerase enzymes suitable for the practice of the present invention are well known in the art and can be derived from a number of sources. Thermostable polymerases may be obtained, for example, from a variety of thermophilic bacteria that are commercially available (for example, from American Type Culture Collection, Rockville, Md.) using methods that are well-known to one of ordinary skill in the art (See, e.g., U.S. Pat. No. 6,245,533). Bacterial cells may be grown according to standard microbiological techniques, using culture media and incubation conditions suitable for growing active cultures of the particular species that are well-known to one of ordinary skill in the art (See, e.g., Brock, T. D., and Freeze, H., J. Bacteriol. 98(1):289-297 (1969); Oshima, T., and Imahori, K, Int. J. Syst. Bacteriol. 24(1):102-112 (1974)). Suitable for use as sources of thermostable polymerases are the thermophilic bacteria Thermus aquaticus, Thermus thermophilus, Thermococcus litoralis, Pyrococcus furiosus, Pyrococcus woosii and other species of the Pyrococcus genus, Bacillus stearothermophilus, Sulfolobus acidocaldarius, Thermoplasma acidophilum, Thermus flavus, Thermus ruber, Thermus brockianus, Thermotoga neapolitana, Thermotoga maritima and other species of the Thermotoga genus, and Methanobacterium thermoautotrophicum, and mutants of each of these species. Preferable thermostable polymerases can include, but are not limited to, Taq DNA polymerase, Tne DNA polymerase, Tma DNA polymerase, or mutants, derivatives or fragments thereof.

Various Sources and/or Preparation Methods of Nucleic Acids

Sources of nucleic acid samples in the disclosed compositions, methods and/or kits include, but are not limited to, human cells such as embryonic stem cells (ESCs), circulating blood, buccal epithelial cells, cultured cells and tumor cells. Also other mammalian tissue, blood and cultured cells are suitable sources of template nucleic acids. In addition, viruses, bacteriophage, bacteria, fungi and other micro-organisms can be the source of nucleic acid for analysis. The DNA may be genomic or it may be cloned in plasmids, bacteriophage, bacterial artificial chromosomes (BACs), yeast artificial chromosomes (YACs) or other vectors. RNA may be isolated directly from the relevant cells or it may be produced by in vitro priming from a suitable RNA promoter or by in vitro transcription. The present invention may be used for the detection of variation in genomic DNA whether human, animal or other. It finds particular use in the analysis of inherited or acquired diseases or disorders. A particular use is in the detection of inherited diseases.

In some embodiments, template sequence or nucleic acid sample can be gDNA. In other embodiments, the template sequence or nucleic acid sample can be cDNA. In yet other embodiments, as in the case of simultaneous analysis of gene expression by RT-PCR, the template sequence or nucleic acid sample can be RNA. The DNA or RNA template sequence or nucleic acid sample can be extracted from any type of tissue including, for example, formalin-fixed paraffin-embedded tumor specimens.

### Preamplification

In some embodiments, additional compositions, methods and kits are provided for “boosting” cast-PCR amplification reactions for limited quantity specimens having very low nucleic acid copy number. In some embodiments, said compositions, methods and kits involve a two-step amplification process comprising a first “booster” or pre-amplification multiplex reaction (see, for example, U.S. Pat. Nos. 6,605,451 and 7,087,414 and U.S. Published Application No. 2004/0175733, the disclosures of which are herein incorporated by reference in their entireties), followed by a second single-plex (i.e., cast-PCR) amplification reaction.

In some preferred embodiments, the first step involves a multiplex reaction which uses at least two complete sets of primers (e.g., one forward allele-1-specific primer, one forward allele-2-specific primer and one reverse locus-specific primer), each set of which is suitable or operative for amplifying a specific polynucleotide of interest. In other embodiments, the resultant multiplex products acquired in the first step are divided into optimized secondary single-plex cast-PCR amplification reactions, each containing at least one primer set previously used in the first multiplexing step and then PCR amplified using the cast-PCR methods described herein.

In other preferred embodiments, the first multiplex reaction is a cast-PCR amplification reaction (although other well known amplification methods such as, but not limited to PCR, RT-PCR, NASBA, SDA, TMA, CRCA, Ligase Chain Reaction, etc. can be used). In certain embodiments, the first multiplex reaction comprises a plurality of allele-specific primers, and locus-specific primers, each group of which is specific for a particular allele of interest and designed according to the cast-PCR methods described herein. Unlike single-plex cast-PCR reactions that generate a single amplified sequence, multiplex cast-PCR amplification reactions, by virtue of utilizing a plurality of different primer sets, can permit the simultaneous amplification of a plurality of different sequences of interest in a single reaction. Because a plurality of different sequences is amplified simultaneously in a single reaction, the multiplex amplifications can effectively increase the concentration or quantity of a sample available for downstream cast-PCR assays. Thus, in some preferred embodiments, significantly more analyses or assays can be performed with a pre-amplified cast-PCR sample than could have been performed with the original sample.

The number of different amplification primer pairs utilized in the multiplex amplification is not critical and can range from as few as two, to as many as tens, hundreds, thousands, or even more. Thus, depending upon the particular conditions, the multiplex amplifications permit the simultaneous amplification of from as few as two to as many as tens, hundreds, thousands, or even more polynucleotide sequences of interest.

The number of amplification cycles performed with a multiplex amplification may depend upon, among other factors, the degree of amplification desired. The degree of amplification desired, in turn, may depend upon such factors as the amount of polynucleotide sample to be amplified or the number of alleles or mutations to be detected using subsequent cast-PCR assays.

In preferred embodiments, it may be desirable to keep the multiplex amplification from progressing beyond the exponential phase or the linear phase. Indeed, in some embodiments, it may be desirable to carry out the multiplex amplification for a number of cycles suitable to keep the reaction within the exponential or linear phase. Utilization of a truncated multiplex amplification round can result in a sample having a boosted product copy number of about 100-1000 fold increase.

In many embodiments, pre-amplification permits the ability to perform cast-PCR assays or analyses that require more sample, or a higher concentration of sample, than was originally available. For example, after a 10×, 100×, 1000×, 10,000×, and so on, multiplex amplification, subsequent cast-PCR single-plex assays can then be performed using, respectively, a 10×, 100×, 1000×, 10,000×, and so on, less sample volume. In some embodiments, this allows each single-plex cast-PCR reaction to be optimized for maximum sensitivity and requires only one method of detection for each allele analyzed. This can be a significant benefit to cast-PCR analysis since, in some embodiments, it allows for the use of off-the-shelf commercially available cast-PCR reagents and kits to be pooled together and used in a multiplex amplification reaction without extensive effort toward or constraints against redesigning and/or re-optimizing cast-PCR assays for any given target sequence. Moreover, in some embodiments, the ability to carry out a multiplex amplification with reagents and kits already optimized for cast-PCR analysis permits the creation of multiplex amplification reactions that are ideally correlated or matched with subsequent single-plex cast-PCR assays.

The following examples are intended to illustrate but not limit the invention.

### EXAMPLES

### Example 1

### General Methylation Specific (MeS) castPCR Assay Design

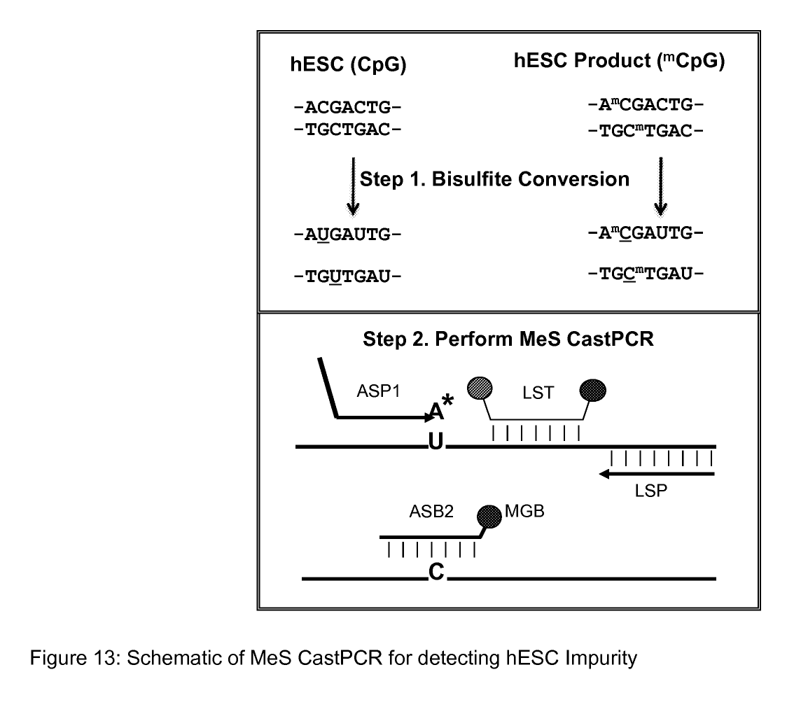

The general schema for the MeS castPCR assays used in the following examples is illustrated in FIG. 13. For each CpG site that is analyzed, allele-specific primers (ASPS) are designed to target a methylated allele (i.e. allele-1) and an unmethylated allele (i.e. allele-2). The castPCR assay reaction mixture for allele-1 analysis includes a 5′-tailed allele-1-specific primer (ASP1), one MGB allele-2 blocker probe (MGB2), one common locus-specific TAQMAN probe (LST) and one common locus-specific primer (LSP). The castPCR assay reaction mixture for analysis of allele-2 includes a 5′-tailed allele-2-specific primer (ASP2), one MGB allele-1 blocker probe (MGB1), one common locus-specific TAQMAN probe (LST) and one common locus-specific primer (LSP). Based on information from Laurent et al., we identified various sequences (Table 1), showing the methylation status of human embryonic stem cells (hESCs), e-fibroblasts (e-fib), and fibroblasts (fib) (Laurent, L. et al., Genome Res. 20: 320-331 (2010). In the methylation-specific castPCR assay, allele-1 is equivalent to detecting uracil-containing sequences (i.e., cytosines converted to uracils during bisulfite conversion), and allele-2 is equivalent to cytosine-containing sequences (i.e., methylcytosines that remain methylcytosines during bisulfite conversion).

TABLE 1

| | | | | | % | % | % | cov- | cov- | cov- | |
|---|---|---|---|---|---|---|---|---|---|---|---|
| | | | | sequence | meth | meth | Meth | erage | erage | erage | 5 mC |
| No. | gene | coordinates | Strand | context | hESC | E-Fib | Fib. | hESC | E-Fib | Fib. | reads |
| 1 | HOXA7- | chr7:27165559 | R | TACACGACAG | 0.0% | 100 | 100 | 9x | 7x | 9x | 6x |
| | HOXA9 | | | | | | | | | | |
| | IGR | | | | | | | | | | |
| 2 | within | chr3:46592433 | R | GCTCCGCAAA | 0.0 | 93 | 100 | 17x | 15x | 11x | 11x |
| | LRRC2 | | | | | | | | | | |
| 3 | within | chr3:46592548, | F | GGCGAAGCGCA | 0.0 | 100 | 100 | 13x | 6x | 5x | 3x |
| | LRRC2 | 53 | | | | | | | | | |
| 4 | within | chr3:46592689, | F | GGGCGCGGTA | 0.0 | 100 | 100 | 16x | 10x | 7x | 3x |
| | LRRC2 | 91 | | | | | | | | | |
| 5 | within | chr17:43983359 | F | GTAGCGGTTA | 0.0 | No data | 100 | 22x | 22x | 21x | 4x |
| | HOXB3 | | | | | | | | | | |
| 6 | within | chr17:43983710 | R | CGGGCGGTGG | 0.0 | 0.0 | 100 | 7x | 1x | 4x | 3.8x |
| | HOXB3 | | | | | | | | | | |
| 7 | within | chr17:43983725 | R | TGTCCGCTAA | 0.0 | 0.0 | 100 | 9x | 1x | 9x | 8x |
| | HOXB3 | | | | | | | | | | |
| 8 | within | chr17:43983772 | R | GCAGCGAAAG | 0.0 | 0.0 | 100 | 15x | 9x | 5x | 4x |
| | HOXB3 | | | | | | | | | | |
| 9 | within | chr17:43984091 | R | GAGTCGTCAA | 0.0 | 32.0 | 100 | 19x | 13x | 5x | 4x |
| | HOXB3 | | | | | | | | | | |
| 10 | within | chr17:43984112, | F | AGCGCCTCGTC | 10.0 | 100 | 100 | 21x | 11x | 7x | 3x |
| | HOXB3 | 17 | | | | | | | | | |
| 11 | within | chr17:43984113, | R | GACGAGGCGCT | 0 | 76 | 100 | 21x | 11x | 7x | 4x |
| | HOXB3 | 18 | | | | | | | | | |
| 12 | within | chr17:44033394- | R | CTCCCACACAC | no | 100 | 100 | 30x | 18x | 25x | 7x |
| | HOXB6 | 44033400 | | | data | | | | | | |

Reaction Conditions: Each assay reaction mixture (10 μl total) contained 1× TaqMan Genotyping Master Mixture (Applied Biosystems, Foster City, Calif.; P/N 437135), 0-10 ng of bisulfite converted genomic DNA (or as indicated otherwise), 300 nM (unless specified otherwise) tailed-, or in some cases untailed-, allele-specific primer (ASP1 for detection of allele-1 or ASP2 for detection of allele-2), 200 nM TaqMan probe (LST), 900 nM locus-specific primer (LSP), 150 nM allele-specific MGB blocker probe (MGB1 for detection of allele-2 or MGB2 for detection of allele-1). The reactions were incubated in a 384-well plate at 95° C. for 10 minutes, then for 5 cycles at 95° C. for 15 seconds and 58° C. for 1 minute, then by 45 cycles at 95° C. for 15 seconds and 60° C. for 1 minute. All reactions were run in triplicates in an ABI PRISM 7900HT® Sequence Detection System, according to the manufacturer's instructions.

The 2-stage cycling protocol used in the following examples for cast-PCR amplification reactions is different from conventional allele-specific PCR. The 2-stage cycling protocol comprises an initial 5 cycles at a lower annealing/extension temperature (e.g., 58° C.), followed by 45 standard cycles at a higher annealing/extension temperature (e.g., 60° C.). Due to the lower Tm of cast-PCR allele-specific primers (e.g., 53-56° C.), PCR is not optimal at standard annealing/extension conditions (e.g., 60° C.). Consequently, lower annealing/extension temperatures used during the initial 5 cycles increases overall cast-PCR efficiency.

Nucleic Acid Samples: Genomic DNA samples were purchased from Coriell Institute for Medical Research (Camden, N.J.) or Zymo Research (Irvine, Calif.).

Target sequence, probes and primers for HOXB3 (chr17:43983725) (sequence number 7 in Table 1).

Target sequence:

GGATAAGGGGGAGAAAGCACGCACGCGAAAATCCAGCCCTAAATAAA
TGGCCATGCGGCTCTGTCTGCGTGACATGATCAAATTTTCATAAGCT
GGGGCAGCGAAAGGAGAACAGGTCTCTTAATAAATGCCACTATTATG
AGAGTGTCCGCTAATGCGACGGGCGGTGGGGTGGGGGAGTGAGGAGG
CGGCGGGGACCCGAAGGGAGGGCGGCGGGGGCTCTGAGTCCAGGCCT
GGATTTATTAAGAAACGATGCATTCAATTTCGGCGTGTTCAGTAATT
ATCTTTTATTTCATTTTC.
(target sequence in bold)

After Bisulfite Conversion

Methylated

ACGTACGCGAAAATTTAGTTTTAAATAAATGGTTATGCGGTTTTGT
TTGCGTGATATGATTAAATTTTTATAAGTTGGGGTAGCGAAAGGAG
AATAGGTTTTTTAATAAATGTTATTATTATAGAGTGTTCGTTAATG
CGACGGGCGGTGGGGT
(target sequence in bold, modified sequences are
underlined)

Unmethylated

ATGTATGTGAAAATTTAGTTTTAAATAAATGGTTATGTGGTTTTGTTT
GTGTGATATGATTAAATTTTTATAAGTTGGGGTAGTGAAAGGAGAATA
GGTTTTTTAATAAATGTTATTATTATAGAGTGTTTGTTAATGTGATGG
GTGGTGGGGT
(target sequence in bold, modified sequences are
underlined)

Probes and Primers

mASP: GCCCCGTCGCATTAACG
uASP: TGCCCACCCATCACATTAACA
LSP: GGTTATGTGGTTTTGTTTGTGTGATATGATTA
LST: (6-FAM)TTGGGGTAGTGAAAGGAGAATAGGT(MGB)
mASB: CATTAACGAACACTC
uASB: CATTAACAAACACTCTA

Bisulfite conversion of genomic DNA samples: For differential detection of methylated and unmethylated cytosines of CpG dinucleotides, genomic DNA samples were bisulfite converted using the protocol of Cells-to-CpG™ Bisulfite Conversion Kit from Life Technologies (Carlsbad, Calif.). Briefly, 2 μg gDNA was mixed with Denaturation Reagent and incubated at 50° C. for 10 minutes. Denatured gDNAs were mixed with Conversion Reagent at the cycling conditions of 95° C. for 3 minutes, 65° C. for 60 minutes, 95° C. for 3 minutes, 65° C. for 30 minutes, and then 4° C. for up to 4 hours. The converted gDNA samples were desalted by using a column containing Binding Buffer and followed by Washing Buffer. Then Desulfonation Reagent was added to the column and incubated at room temperature for 15 minutes. Column was spun and washed, and gDNA samples were eluted using 40 μl Elution Buffer into new tubes. The converted DNA samples were quantified by Nanodrop (Thermo Scientific, Wilmington, Del.). Conversion recovery rate and efficiency were evaluated according to the manufacturer's recommendation.

Data Analysis: An automatic baseline and manual threshold of 0.2 were used to calculate the threshold cycle (Cₜ) which is defined as the fractional cycle number at which the fluorescence passes the fixed threshold. PCR reactions were run for a total of 50 cycles. For castPCR reactions, there was a pre-run of five cycles at a lower annealing/extension temperature followed by an additional 45 cycles at a higher annealing/extension temperature.

Experimental Design:

Specificity of MeS castPCR

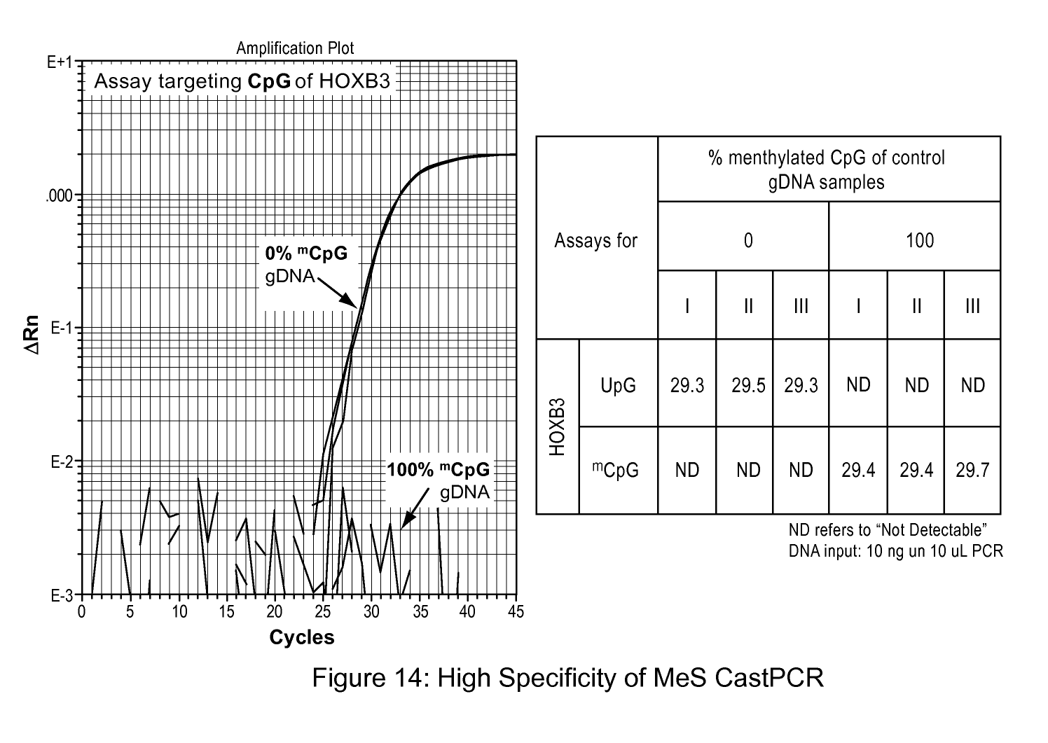

In this example in FIG. 14, the castPCR assay specifically targeting ^{m}CpG site of HOXB3 gene detect fully (100%) methylated gDNA. No signal was observed in unmethylated (0%) gDNA. A total of 10 ng bisulfite converted gDNA was added in each of 10 μl_PCR reactions.

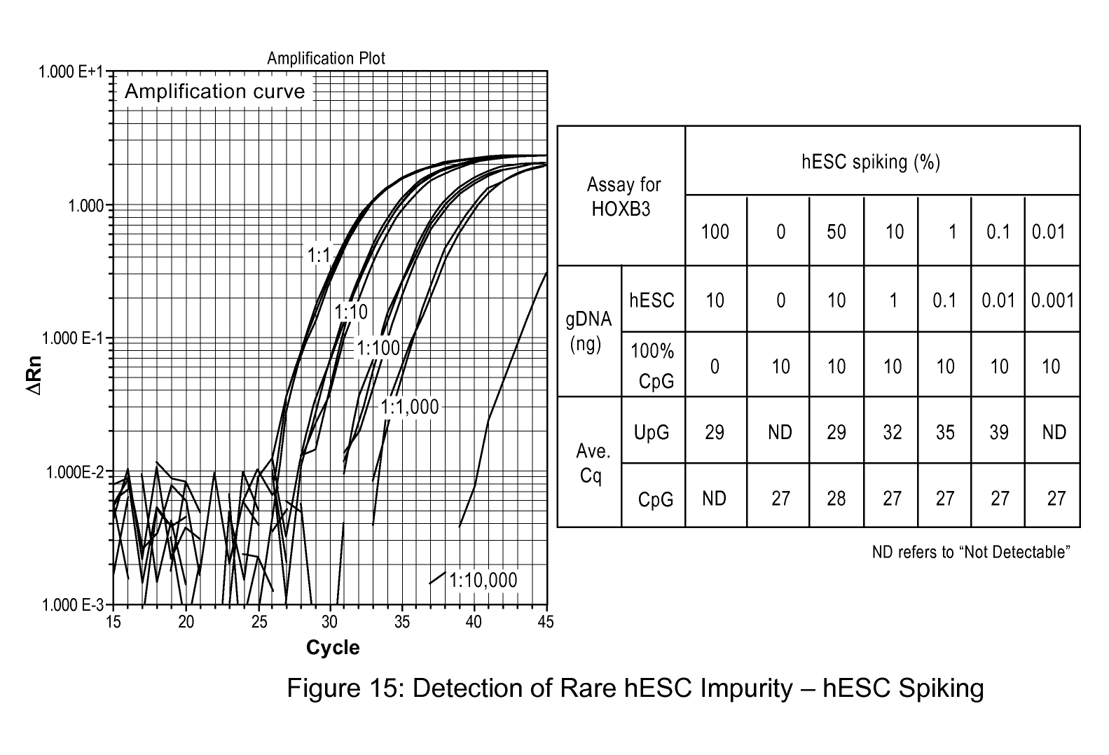

In this example in FIG. 15, the selectivity of castPCR, i.e., the ability of MeS castPCR to detect gDNA (up to a single copy) from spike-in hESC cells in the presence of fully methylated gDNA (thousands of copies), was determined.

FIG. 15 shows that castPCR is able to detect as little 10 μg (^{˜}3 copies) of an unmethylated DNA sequence (from spike-in embryonic stem cells, for example), even when surrounded by a 1.000-fold excess of fully methylated DNA sequences.

### Example 2

Similar to Example 1, probes and primers are derived for other sequences shown in Table 1 and reactions are performed to determine the presence or absence of methylcytosine in a nucleic acid containing sample.

### Example 3

Similar to Examples above, an RNA containing sample is analyzed to determine presence or absence of methylcytosine. In this experiment, the RNA-containing sample is divided into at least two reactions. One portion is treated with bisulfite and then reverse transcribed, while the other portions is reverse transcribed without the bisulfite treatment. The two portions of the samples are further processed as above for Example 1 with suitable primers and probes.

We claim:

1. A method for detecting at least one first unmethylated cytosine allelic variant of a target sequence in a nucleic acid sample suspected of also comprising at least one methylated cytosine allelic variant of the target sequence, comprising:
a) bisulfite converting an aliquot of a nucleic acid sample;
b) forming a reaction mixture by combining:
I. the bisulfite converted nucleic acid sample aliquot;
II. a first allele-specific primer, wherein an unmethylated cytosine allele-specific nucleotide portion of the first allele-specific primer is complementary to the first uracil-containing allelic variant of the target sequence;
III. a first allele-specific blocker probe, wherein a cytosine allele-specific nucleotide portion of the first allele-specific primer is complementary to the first methylcytosine-containing allelic variant of the target sequence, and wherein the first allele-specific blocker probe comprises a minor groove binder;
IV. a first locus-specific primer that is complementary to a region of the target sequence that is 3′ from the first allelic variant and on the opposite strand; and
V. a first detector probe;
c) carrying out an amplification reaction; and
d) detecting the first amplicon by detecting a change in a detectable property of the first detector probe, thereby detecting the first unmethylated cytosine allelic variant of the target gene in the nucleic acid sample.

2. The method of claim 1, further comprising using the change in a detectable property of the first detector probe to quantitate the first allelic variant.

3. A method according to claim 1, further comprising:
a) taking a second aliquot of the nucleic acid sample;
b) forming a reaction mixture by combining:
i) the second nucleic acid sample aliquot;
ii) a second allele-specific primer, wherein a cytosine allele-specific nucleotide portion of the second allele-specific primer is complementary to the second methylated cytosine-containing allelic variant of the target sequence;
iii) a second allele-specific blocker probe, wherein a uracil allele-specific nucleotide portion of the second allele-specific primer is complementary to the second cytosine-containing allelic variant of the target sequence, and wherein the second allele-specific blocker probe comprises a minor groove binder;
iv) a second locus-specific primer that is complementary to a region of the target sequence that is 3′ from the second allelic variant and on the opposite strand; and
v) a second detector probe;
c) carrying out an amplification reaction; and
d) detecting the second amplicon by detecting a change in a detectable property of the second detector probe, thereby detecting the second methylated cytosine allelic variant of the target gene in the nucleic acid sample.

4. The method of claim 3, further comprising comparing the change in a detectable property of the first detector probe in the first reaction mixture to the change in a detectable property of the second detector probe in the second reaction mixture.

5. The method of claim 1 or 3, wherein said first, second or first and second allele-specific primer and/or said first, second, or first and second allele-specific blocker probe comprises at least one modified base.

6. The method of claim 5, wherein said modified base is an 8-aza-7-deaza-dN (ppN) base analog, where N is adenine (A), cytosine (C), guanine (G), or thymine (T).

7. The method of claim 5, wherein said modified base is a locked nucleic acid (LNA) base.

8. The method of claim 5, wherein said modified base is a fdU or iso dC base.

9. The method of claim 5, wherein said modified base is any modified base that increases the Tm between matched and mismatched target sequences or nucleotides.

10. The method of claim 5, wherein said modified base is located at (a) the 3′-end, (b) the 5′-end, (c) at an internal position or at any combination of (a), (b) or (c) within said allele-specific primer and/or allele-specific blocker probe.

11. The method of claim 5, wherein the specificity of said detecting is improved by the inclusion of said modified base in said first, second or first and second allele-specific primer and/or said first, second, or first and second allele-specific blocker probe as compared to when it is not.

12. The method of claim 11, wherein said improvement is at least 2 fold.

13. The method of claim 1 or 3, wherein the specificity of said detecting is improved by at least 2 fold as compared to the specificity of detecting an allelic variant in a nucleic acid sample using ASB-PCR methods.

14. The method of claim 1 or 3, wherein said carrying out an amplification reaction comprises a 2-stage cycling protocol.

15. The method of claim 14, wherein the number of cycles in the first stage of said 2-stage cycling protocol comprises fewer cycles than the number of cycles used in the second stage.

16. The method of claim 14, wherein said number of cycles in the first stage is about 90% fewer cycles than said number of cycles in the second stage.

17. The method of claim 14, wherein said number of cycles in the first stage is between 3-7 cycles and said number of cycles in the second stage is between 25-48 cycles.

18. The method of claim 14, wherein the annealing/extension temperature used during the first cycling stage of said 2-stage cycling protocol is between 1-7° C. lower than the annealing/extension temperature used during the second stage.

19. The method of claim 14, wherein said annealing/extension temperature used during the first cycling stage of said 2-stage cycling protocol is between 56-59° C. and said annealing/extension temperature used during said second stage is between 60-65° C.

20. The method of claim 1, wherein said step (b) is preceded by a pre-amplification step.

21. The method of claim 20, wherein said pre-amplification step comprises a multiplex amplification reaction that uses at least two complete sets of allele-specific primers and locus-specific primers, wherein each set is suitable or operative for amplifying a specific polynucleotide of interest.

22. The method of claim 21, wherein the products of said multiplex amplification reaction are divided into secondary single-plex amplification reactions, wherein each single-plex amplification reaction contains at least one primer set previously used in said multiplex reaction.

23. The method of claim 21, wherein said multiplex amplification reaction further comprises a plurality of allele-specific blocker probes.

24. The method of claim 21, wherein said multiplex amplification reaction is carried out for a number of cycles suitable to keep the reaction within the linear phase of amplification.

25. A method for detecting undifferentiated hESCs in a cell population, comprising the steps of:
a) bisulfite converting a nucleic acid sample isolated from said cell population;
b) optionally performing multiplex preamplification of said nucleic acid sample;
c) performing castPCR of said nucleic acid sample; and
d) determining if said nucleic acid sample is unmethylated at specific loci of nucleic acids to detect the presence of undifferentiated hESCs in said cell population.

26. A method according to claim 25, wherein said nucleic acid sample is from a population of cells comprising undifferentiated hESCs.

27. The method of claim 25, wherein said nucleic acid sample is from a population of cells comprising differentiated hESCs.

28. The method of claim 27, wherein said differentiated hESCs comprise terminally differentiated cells.

29. A method for detecting fetal cells in a cell population, comprising the steps of:
a) bisulfite converting a nucleic acid sample isolated from said cell population;
b) optionally performing multiplex preamplification of said nucleic acid sample;
c) performing castPCR of said nucleic acid sample; and
d) determining if said nucleic acid sample is unmethylated at specific loci of nucleic acids to detect the presence of fetal cells in said cell population.

30. A composition for detecting at least one first unmethylated cytosine allelic variant of a target sequence in a nucleic acid sample suspected of also comprising at least one methylated cytosine allelic variant of the target sequence, comprising:
a) a reagent for bisulfite converting an aliquot of a nucleic acid sample;
b) a first allele-specific primer, wherein an unmethylated cytosine allele-specific nucleotide portion of the first allele-specific primer is complementary to the first uracil-containing allelic variant of the target sequence;
c) a first allele-specific blocker probe, wherein a cytosine allele-specific nucleotide portion of the first allele-specific primer is complementary to the first methylcytosine-containing allelic variant of the target sequence, and wherein the first allele-specific blocker probe comprises a minor groove binder;
d) a first locus-specific primer that is complementary to a region of the target sequence that is 3′ from the first allelic variant and on the opposite strand; and
e) a first detector probe.

31. A kit for detecting at least one first unmethylated cytosine allelic variant of a target sequence in a nucleic acid sample suspected of also comprising at least one methylated cytosine allelic variant of the target sequence, comprising:
a) a reagent for bisulfite converting an aliquot of a nucleic acid sample;
b) a first allele-specific primer, wherein an unmethylated cytosine allele-specific nucleotide portion of the first allele-specific primer is complementary to the first uracil-containing allelic variant of the target sequence;
c) a first allele-specific blocker probe, wherein a cytosine allele-specific nucleotide portion of the first allele-specific primer is complementary to the first methylcytosine-containing allelic variant of the target sequence, and wherein the first allele-specific blocker probe comprises a minor groove binder;
d) a first locus-specific primer that is complementary to a region of the target sequence that is 3′ from the first allelic variant and on the opposite strand; and
e) a first detector probe.


SEQUENCE LISTING (21 sequences)

SEQ ID NO: 1 - DNA, 10 bp, Homo sapiens
tacacgacag

SEQ ID NO: 2 - DNA, 10 bp, Homo sapiens
gctccgcaaa

SEQ ID NO: 3 - DNA, 11 bp, Homo sapiens
ggcgaagcgca

SEQ ID NO: 4 - DNA, 10 bp, Homo sapiens
gggcgcggta

SEQ ID NO: 5 - DNA, 10 bp, Homo sapiens
gtagcggtta

SEQ ID NO: 6 - DNA, 10 bp, Homo sapiens
cgggcggtgg

SEQ ID NO: 7 - DNA, 10 bp, Homo sapiens
tgtccgctaa

SEQ ID NO: 8 - DNA, 10 bp, Homo sapiens
gcagcgaaag

SEQ ID NO: 9 - DNA, 10 bp, Homo sapiens
gagtcgtcaa

SEQ ID NO: 10 - DNA, 11 bp, Homo sapiens
agcgcctcgtc

SEQ ID NO: 11 - DNA, 11 bp, Homo sapiens
gacgaggcgct

SEQ ID NO: 12 - DNA, 11 bp, Homo sapiens
ctcccacacac

SEQ ID NO: 13 - DNA, 300 bp, Homo sapiens
ggataagggggagaaagcacgcacgcgaaaatccagccctaaataaatggccatgcggctctgtctgcgtgacatgatcaaattttcataagctggggcagcgaaaggagaacaggtctcttaataaatgccactattatagagtgtccgctaatgcgacgggcggtggggtgggggagtgaggaggcggcggggacccgaaggggagggcggcgggggctctgagtccaggcctggatttattaagaaacgatgcattcaatttcggcgtgttcagtaattatcttttatttcattttc

SEQ ID NO: 14 - DNA, 154 bp, Artificial Sequence
  Description of Artificial Sequence Synthetic polynucleotide
acgtacgcgaaaatttagttttaaataaatggttatgcggttttgtttgcgtgatatgattaaatttttataagttggggtagcgaaaggagaataggttttttaataaatgttattattatagagtgttcgttaatgcgacgggcggtggggt

SEQ ID NO: 15 - DNA, 154 bp, Artificial Sequence
  Description of Artificial Sequence Synthetic polynucleotide
atgtatgtgaaaatttagttttaaataaatggttatgtggttttgtttgtgtgatatgattaaatttttataagttggggtagtgaaaggagaataggttttttaataaatgttattattatagagtgtttgttaatgtgatgggtggtggggt

SEQ ID NO: 16 - DNA, 17 bp, Artificial Sequence
  Description of Artificial Sequence Synthetic primer
gccccgtcgcattaacg

SEQ ID NO: 17 - DNA, 21 bp, Artificial Sequence
  Description of Artificial Sequence Synthetic primer
tgcccacccatcacattaaca

SEQ ID NO: 18 - DNA, 32 bp, Artificial Sequence
  Description of Artificial Sequence Synthetic primer
ggttatgtggttttgtttgtgtgatatgatta

SEQ ID NO: 19 - DNA, 25 bp, Artificial Sequence
  Description of Artificial Sequence Synthetic probe
ttggggtagtgaaaggagaataggt

SEQ ID NO: 20 - DNA, 15 bp, Artificial Sequence
  Description of Artificial Sequence Synthetic probe
cattaacgaacactc

SEQ ID NO: 21 - DNA, 17 bp, Artificial Sequence
  Description of Artificial Sequence Synthetic probe
cattaacaaacactcta